(19) United States
(12) Patent Application Publication
(10) Pub. No.: US 2009/0105234 A1
(43) Pub. Date: Apr. 23, 2009

(54) EP4 Receptor Agonist, Compositions and Methods Thereof

(76) Inventors: John Colucci, Kirkland (CA); Yongxin Han, Kirkland (CA); Julie A. Farand, Montreal (CA)

Correspondence Address: MERCK AND CO., INC, P O BOX 2000, RAHWAY, NJ, 07065-0907, US

(21) Appl. No.: 11/989,643

(22) PCT Filed: Jul. 28, 2006
(86) PCT No.: PCT/CA2006/001243

§ 371 Date: Jan. 28, 2008

Related U.S. Application Data
(60) Provisional application No. 60/705,124, filed on Aug. 3, 2005.

(51) Int. Cl. A61K 31/5355; C07D 413/06; A61P 27/02

(52) U.S. Cl. 514/228.8; 544/96

(57) ABSTRACT

This invention relates to potent selective agonists of the EP₄ subtype of prostaglandin E2 receptors, their use or a formulation thereof in the treatment of glaucoma and other conditions, which are related to elevated intraocular pressure in the eye of a patient. This invention further relates to the use of the compounds of this invention for mediating the bone modeling and remodeling processes of the osteoblasts and osteoclasts.

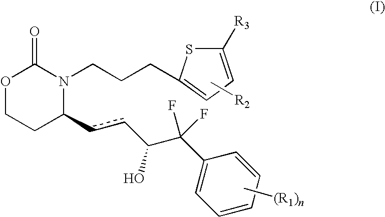

### BACKGROUND OF THE INVENTION

Glaucoma is a degenerative disease of the eye wherein the intraocular pressure is too high to permit normal eye function. As a result, damage may occur to the optic nerve head and result in irreversible loss of visual function. If untreated, glaucoma may eventually lead to blindness. Ocular hypertension, i.e., the condition of elevated intraocular pressure without optic nerve bead damage or characteristic glaucomatous visual field defects, is now believed by the majority of ophthalmologists to represent merely the earliest phase in the onset of glaucoma.

Many of the drugs formerly used to treat glaucoma proved unsatisfactory. Current methods of treating glaucoma include using therapeutic agents such as pilocarpine, carbonic anhydrase inhibitors, beta-blockers, prostaglandins and the like. However, these therapies often produce undesirable local effects. As can be seen, there are several current therapies for treating glaucoma and elevated intraocular pressure, but the efficacy and the side effect profiles of these agents are not ideal. Therefore, there still exists the need for new and effective therapies with little or no side effects.

A variety of disorders in humans and other mammals involve or are associated with abnormal or excessive bone loss. Such disorders include, but are not limited to, osteoporosis, glucocorticoid induced osteoporosis, Paget's disease, abnormally increased bone turnover, periodontal disease, tooth loss, bone fractures, rheumatoid arthritis, periprosthetic osteolysis, osteogenesis imperfecta, metastatic bone disease, hypercalcemia of malignancy, and multiple myeloma. One of the most common of these disorders is osteoporosis, which in its most frequent manifestation occurs in postmenopausal women. Prostaglandins such as the PGE₂ series are known to stimulate bone formation and increase bone mass in mammals, including man. It is believed that the four different receptor subtypes, designated EP₁, EP₂, EP₃, and EP₄ are involved in mediating the bone modeling and remodeling processes of the osteoblasts and osteoclasts. The major prostaglandin receptor in bone is EP₄, which is believed to provide its effect by signaling via cyclic AMP. In the present invention it is found that the formula I agonists of the EP₄ subtype receptor are useful for stimulating bone formation. WO 02/24647, WO 02/42268, EP 1114816, WO 01/46140 and WO 01/72268 disclose EP₄ agonists. However, they do not disclose the compounds of the instant invention.

### SUMMARY OF THE INVENTION

This invention relates to agonists of the EP₄ subtype of prostaglandin E2 receptors and their use or a formulation thereof in the treatment of glaucoma and other conditions that are related to elevated intraocular pressure in the eye of a patient. In particular, this invention relates to a series of 3,4-disubstituted 1,3-oxazinan-2-one, derivatives and their use to treat ocular diseases and to provide a neuroprotective effect to the eye of mammalian species, particularly humans. This invention further relates to the use of the compounds of this invention for mediating the bone modeling and remodeling processes of the osteoblasts and osteoclasts.

More particularly, this invention relates to novel EP₄ agonist having the structural formula I:

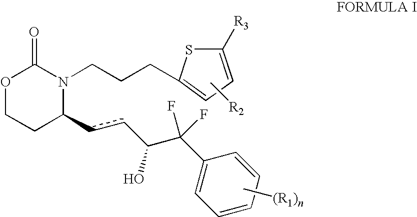

or a pharmaceutically acceptable salt, enantiomer, diastereomer, prodrug or mixture thereof, wherein,
R and R₄ independently represent H, or C₁₋₆ alkyl;
R₁ independently represents hydrogen, C₁₋₆ alkyl, halogen, CF₃, aryl, said aryl optionally substituted with 1 to 3 groups of halogen, C₁₋₆ alkyl, CF₃, or N(R₄)₂;
R₂ represents H, or halogen;
R₃ represents COOR or carboxylic acid isostere;
n represents 0-3; and
- - - represents a double or single bond.

This and other aspects of the invention will be realized upon inspection of the invention as a whole.

### DETAILED DESCRIPTION OF THE INVENTION

The invention is described herein in detail using the terms defined below unless otherwise specified.

The term “therapeutically effective amount”, as used herein, means that amount of the EP₄ receptor subtype agonist of formula I, or other actives of the present invention, that will elicit the desired therapeutic effect or response or provide the desired benefit when administered in accordance with the desired treatment regimen. A preferred therapeutically effective amount relating to the treatment of abnormal bone resorption is a bone formation, stimulating amount. Likewise, a preferred therapeutically effective amount relating to the treatment of ocular hypertension or glaucoma is an amount effective for reducing intraocular pressure and/or treating ocular hypertension and/or glaucoma.

“Pharmaceutically acceptable” as used herein, means generally suitable for administration to a mammal, including humans, from a toxicity or safety standpoint.

The term “prodrug” refers to compounds which are drug precursors which, following administration and absorption, release the claimed drug in vivo via some metabolic process. A non-limiting example of a prodrug of the compounds of this invention would be an ester of the carboxylic acid group, where the ester functionality is easily hydrolyzed to the active acid after administration to a patient. Exemplary prodrugs include acetic acid derivatives that are non-narcotic, analgesics/non-steroidal, anti-inflammatory drugs having a free CH₂COOH group (which can optionally be in the form of a pharmaceutically acceptable salt, e.g. —CH₂COO—Na⁺), typically attached to a ring system, preferably to an aromatic or heteroaromatic ring system.

The term “alkyl” refers to a monovalent alkane (hydrocarbon) derived radical containing from 1 to 6 carbon atoms unless otherwise defined. It may be straight, branched or cyclic. Preferred alkyl groups include methyl, ethyl, propyl, isopropyl, butyl, t-butyl, cyclopentyl and cyclohexyl. When the alkyl group is said to be substituted with an alkyl group, this is used interchangeably with “branched alkyl group”.

Cycloalkyl is a species of alkyl containing from 3 to 6 carbon atoms, without alternating or resonating double bonds between carbon atoms. Examples of cycloalkyl groups are cyclopropyl, cyclobutyl, cyclopentyl, and cyclohexyl.

Halogen (halo) refers to chlorine, fluorine, iodine or bromine.

Carboxylic isostere represents tetrazole, acylsulfonamide, sulfonic acid, phosphonic acid or prodrug such as C₁₋₆ aldehyde or C₁₋₆ alcohol.

Aryl refers to aromatic rings e.g., phenyl, substituted phenyl and the like, as well as rings which are fused, e.g., naphthyl, phenanthrenyl and the like. An aryl group thus contains at least one ring having at least 6 atoms, with up to five such rings being present, containing up to 22 atoms therein, with alternating (resonating) double bonds between adjacent carbon atoms or suitable heteroatoms. Examples of aryl groups are phenyl, naphthyl, tetrahydronaphthyl, indanyl, biphenyl, phenanthryl, anthryl or acenaphthyl and phenanthrenyl, preferably phenyl, naphthyl or phenanthrenyl. Aryl groups may likewise be substituted as defined. Preferred substituted aryls include phenyl and naphthyl.

The term “agonist” as used herein means EP₄ subtype compounds of formula I interact with the EP₄ receptor to produce maximal, super maximal or submaximal effects compared to the natural agonist, PGE2. See Goodman and Gilman, The Pharmacological Basis of Therapeutics, 9^{th} edition, 1996, chapter 2.

One embodiment of this invention is realized when R₃ is COOR and all other variables are as originally desfined.

Another embodiment of this invention is realized when R₃ is a carboxylic acid isostere and all other variables are as originally defined.

Another embodiment of this invention is realized when R is H and all other variables are as originally defined.

Another embodiment of this invention is realized when R is C₁₋₆ alkyl and all other variables are as originally defined. A sub-embodiment of this invention is realized when R is isopropyl, ethyl or, methyl, preferably isopropyl.

Another embodiment of this invention is realized when R₂ is hydrogen and all other variables are as originally defined.

Another embodiment of this invention is realized when R₂ is halogen and all other variables are as originally defined. A subembodiment of this invention is realized when halogen is fluorine or bromine.

Still another embodiment of this invention is realized when R₁ is halogen and all other variables are as originally defined. A sub-embodiment is realized when R₁ is bromine or chlorine, preferably bromine.

Still another embodiment of this invention is realized when R₁ is C₁₋₆ alkyl and all other variables are as originally defined.

Still another embodiment of this invention is realized when R₁ is CF₃ and all other variables are as originally defined.

Another embodiment of this invention is realized when R₃ is COOR, R is H or isopropyl, R₁ is bromine and R₂ is hydrogen.

Another embodiment of this invention is realized when R₃ is tetrazole, R₁ is bromine and R₂ is hydrogen

Another embodiment of this invention is reaslized when n is 0, 1, or 2 and all other variables are as originally defined.

Still another embodiment of this invention is realized when n is 0 and all other variables are as originally defined

Another embodiment of this invention is realized when n is 1 and all other variables are as originally defined. A subembodiment of this invention is realized when R₁ is bromine and R₂ is hydrogen.

Another embodiment of this invention is realized when n is 2 and all other variables are as originally defined.

Another embodiment of this invention is realized when - - - represents a double bond

Compounds of this invention are:
Isopropyl 5-(3-{(4R)-4-[(1E,3R)-4-(3-bromophenyl)-4,4-difluoro-3-hydroxybut-1-en-1-yl]-2-oxo-1,3-oxazinan-3-yl}propyl)thiophene-2-carboxylate;
Isopropyl 5-(3-{(4R)-4-[(1E)-4-(3-bromophenyl)-4,4-difluoro-3-oxobut-1-en-1-yl]-2-oxo-1,3-oxazinan-3-yl}propyl)thiophene-2-carboxylate;
Isopropyl 5-(3-{(4R)-4-[(1E)-4-(3-bromophenyl)-4,4-difluoro-3-hydroxybut-1-en-1-yl]-2-oxo-1,3-oxazinan-3-yl}propyl)thiophene-2-carboxylate;
5-(3-{(4R)-4-[(1E,3R)-4-(3-bromophenyl)-4,4-difluoro-3-hydroxybut-1-en-1-yl]-2-oxo-1,3-oxazinan-3-yl}propyl)thiophene-2-carboxylic acid;
Isopropyl 5-(3-{(4S)-4-[(3R)-4-(3-bromophenyl)-4,4-difluoro-3-hydroxybutyl]-2-oxo-1,3-oxazinan-3-yl}propyl)thiophene-2-carboxylate;
5-(3-{(4S)-4-[(3R)-4-(3-bromophenyl)4,4-difluoro-3-hydroxybutyl]-2-oxo-1,3-oxazinan-3-yl}propyl)thiophene-2-carboxylic acid;
5-(3-{(4R)-4-[(1E)-4,4-difluoro-3-hydroxy-4-phenylbut-1-en-1-yl]-2-oxo-1,3-oxazinan-3-yl}propyl)thiophene-2-carboxylic acid;
Isopropyl 5-(3-{(4R)-4-[(1E,3R)-4-3,5-dichlorophenyl)-4,4-difluoro-3-hydroxybut-1-en-1-yl]-2-oxo-1,3-oxazinan-3-yl}propyl)thiophene-2-carboxylate;
5-(3-{(4R)-4-[(1E,3R)-4-(3,5-dichlorophenyl)-4,4-difluoro-3-hydroxybut-1-en-1-yl]-2-oxo-1,3-oxazinan-3-yl}propyl)thiophene-2-carboxylic acid;
Isopropyl 5-(3-{(4S)-4-[(3R)-4-(3,5-dichlorophenyl)-4,4-difluoro-3-hydroxybutyl]-2-oxo-1,3-oxazinan-3-yl}propyl)thiophene-2-carboxylate;
5-(3-{(4S)-4-[(3R)-4-(3,5-dichlorophenyl)-4,4-difluoro-3-hydroxybutyl]-2-oxo-1,3-oxazinan-3-yl}propyl)thiophene-2-carboxylic acid;
Isopropyl 5-(3-{(4R)-4-[(1E,3R)-4-(3,5-dimethylphenyl)-4,4-difluoro-3-hydroxybut-1-en-1-yl]-2-oxo-1,3-oxazinan-3-yl}propyl)thiophene-2-carboxylate;
5-(3-{(4R)-4-[(1E,3R)-4-(3,5-dimethylphenyl)-4,4-difluoro-3-hydroxybut-1-en-1-yl]-2-oxo-1,3-oxazinan-3-yl}propyl)thiophene-2-carboxylic acid;
Isopropyl 5-(3-{(4S)-4-[(3R)-4-(3,5-dimethylphenyl)-4,4-difluoro-3-hydroxybutyl]-2-oxo-1,3-oxazinan-3-yl}propyl)thiophene-2-carboxylate;
5-(3-{(4S)-4-[(3R)-4-3,5-dimethylphenyl)-4,4-difluoro-3-hydroxybutyl]-2-oxo-1,3-oxazinan-3-yl}propyl)thiophene-2-carboxylic acid;
Isopropyl 5-[3-((4R)-4-{(1E,3R)-4,4-difluoro-3-hydroxy-4-[3-trifluoromethyl)phenyl]but-1-en-1-yl}-2-oxo-1,3-oxazinan-3-yl)propyl]thiophene-2-carboxylate;
5-[3-((4R)-4-{(1E,3R)-4,4-difluoro-3-hydroxy-4-[3-trifluoromethyl)phenyl]but-1-en-1-yl}-2-oxo-1,3-oxazinan-3-yl)propyl]thiophene-2-carboxylic acid;
Isopropyl 5-[3-((4S)-4-{(3R)-4,4-difluoro-3-hydroxy-4-[3-(trifluoromethyl)phenyl]butyl}-2-oxo-1,3-oxazinan-3-yl)propyl]thiophene-2-carboxylate;
5-[3-((4S)-4-{(3R)-4,4-difluoro-3-hydroxy-4-[3-(trifluoromethyl)phenyl]butyl}-2-oxo-1,3-oxazinan-3-yl)propyl]thiophene-2-carboxylic acid;
5-(3-{(4R)-4-[(1E,3R)-4,4-difluoro-3-hydroxy-4-(3-isopropylphenyl)but-1-en-1-yl]-2-oxo-1,3-oxazinan-3-yl}propyl)thiophene-2-carboxylic acid;
5-(3-{(4S)-4-[(3R)-4,4-difluoro-3-hydroxy-4-(3-isopropylphenyl)butyl]-2-oxo-1,3-oxazinan-3-yl}propyl)thiophene-2-carboxylic acid;
Methyl 4-bromo-5-(3-{(4R)-4-[(1E,3R)-4-(3-bromophenyl)-4,4-difluoro-3-hydroxybut-1-en-1-yl]-2-oxo-1,3-oxazinan-3-yl}propyl)thiophene-2-carboxylate;
4-Bromo-5-(3-{(4R)-4-[(1E,3R)-4-(3-bromophenyl)-4,4-difluoro-3-hydroxybut-1-en-1-yl]-2-oxo-1,3-oxazinan-3-yl}propyl)thiophene-2-carboxylic acid;
5-(3-{(4R)-4-[(1E,3R)-4-(3-bromophenyl)-4,4-difluoro-3-hydroxybut-1-en-1-yl]-2-oxo-1,3-oxazinan-3-yl}propyl)-3-fluorothiophene-2-carboxylic acid;
5-(3-{(4S)-4-[(3R)-4-(3-bromophenyl)-4,4-difluoro-3-hydroxybutyl]-2-oxo-1,3-oxazinan-3-yl}propyl)-3-fluorothiophene-2-carboxylic acid;
5-(3-{(4S)-4-[(3R)-3-(4′-chloro-2′-methylbiphenyl-3-yl)-3-hydroxypropyl]-2-oxo-1,3-oxazinan-3-yl}propyl)thiophene-2-carboxylic acid;
Isopropyl 5-(3-{(4R)-4-[(1E,3R)-4-(3-bromo-5-iodophenyl)-4,4-difluoro-3-hydroxybut-1-en-1-yl]-2-oxo-1,3-oxazinan-3-yl}propyl)thiophene-2-carboxylate; and
5-(3-{(4R)-4-[(1E,3R)-4-(3-bromo-5-iodophenyl)-4,4-difluoro-3-hydroxybut-1-en-1-yl]-2-oxo-1,3-oxazinan-3-yl}propyl)thiophene-2-carboxylic acid;
   or a pharmaceutically acceptable salt, enantiomer, diastereomer, prodrug or mixture thereof.

Another embodiment of this invention is directed to a composition containing an EP₄ agonist of Formula I and optionally a pharmaceutically acceptable carrier.

Yet another embodiment of this invention is directed to a method for decreasing elevated intraocular pressure or treating glaucoma by administration, preferably topical or intra-camaral administration, of a composition containing an EP₄ agonist of Formula I and optionally a pharmaceutically acceptable carrier. Use of the compounds of formula I for the manufacture of a medicament for treating elevated intraocular pressure or glaucoma or a combination thereof is also included in this invention

This invention is further concerned with a process for making a pharmaceutical composition comprising a compound of formula I.

This invention is further concerned with a process for making a pharmaceutical composition comprising a compound of formula I, and a pharmaceutically acceptable carrier.

The claimed compounds bind strongly and act on PGE₂ receptor, particularly on the EP₄ subtype receptor and therefore are useful for preventing and/or treating glaucoma and ocular hypertension.

Dry eye is a common ocular surface disease afflicting millions of people. Although it appears that dry eye may result from a number of unrelated pathogenic causes, the common end result is the breakdown of the tear film, which results in dehydration of the exposed outer surface of the eye. (Lemp, Report of the Nation Eye Institute/Industry Workshop on Clinical Trials in Dry Eyes, The CLAO Journel, 21(4):221-231 (1995)). Functional EP4 receptors have been found in human conjuctival epithelial cells (see U.S. Pat. No. 6,344,477, incorporated by reference in its entirey) and it is appreciated that both human corneal epithelial cells (Progess in Retinal and Eye Research, 16:81-98 (1997)) and conjuctival cells (Dartt et al. Localization of nerves adjacent to goblet cells in rat conjucntiva. Current Eye Research, 14:993-1000 (1995)) are capable of secreting mucins. Thus, the compounds of formula I are useful for treating dry eye.

Macular edema is swelling within the retina within the critically important central visual zone at the posterior pole of the eye. It is believed that EP₄ agonist which lower IOP are useful for treating diseases of the macular such as macular edema or macular degeneration. Thus, another aspect of this invention is a method for treating macular edema or macular degeneration.

Glaucoma is characterized by progressive atrophy of the optic nerve and is frequently associated with elevated intraocular pressure (IOP). It is possible to treat glaucoma, however, without necessarily affecting IOP by using drugs that impart a neuroprotective effect. See Arch. Opthalmol. Vol. 112, January 1994, pp. 37-44; Investigative Ophthamol. & Visual Science, 32, 5, April 1991, pp. 1593-99. It is believed that EP₄ agonist which lower IOP are useful for providing a neuroprotective effect. They are also believed to be effective for increasing retinal and optic nerve head blood velocity and increasing retinal and optic nerve oxygen by lowering IOP, which when coupled together benefits optic nerve health. As a result, this invention further relates to a method for increasing retinal and optic nerve head blood velocity, or increasing retinal and optic nerve oxygen tension or providing a neuroprotective effect or a combination thereof by using an EP₄ agonist of formula I.

The compounds produced in the present invention are readily combined with suitable and known pharmaceutically acceptable excipients to produce compositions which may be administered to mammals, including humans, to achieve effective IOP lowering. Thus, this invention is also concerned with compositions and methods of treating ocular hypertension, glaucoma, macular edema, macular degeneration, for increasing retinal and optic nerve head blood velocity, for increasing retinal and optic nerve oxygen tension, for providing a neuroprotective effect or for a combination thereof by administering to a patient in need thereof one of the compounds of formula I alone or in combination with one or more of the following active ingredients, a β-adrenergic blocking agent such as timolol, betaxolol, levobetaxolol, carteolol, levobunolol, a parasympathomimetic agent such as pilocarpine, a sympathomimetic agents such as epinephrine, iopidine, brimonidine, clonidine, para-aminoclonidine, a carbonic anhydrase inhibitor such as dorzolamide, acetazolamide, metazolamide or brinzolamide; COSOPT®, a Maxi-K channel blocker such as Penitrem A, paspalicine, charybdotoxin, iberiotoxin, Paxicillan, Aflitram, Verroculogen, and as disclosed in WO 03/105868 (U.S. Ser. No. 60/389,205), WO 03/105724 (60/389,222), WO 03/105847 (60/458,981), 60/424,790, filed Nov. 8, 2002 (Attorney docket 21260PV), No. 60/424,808, filed Nov. 8, 2002 (Attorney docket 21281 PV), Ser. No. 09/765,716, filed Jan. 17, 2001, Ser. No. 09/764,738, filed Jan. 17, 2001 and PCT publications WO 02/077168 and WO 02/02060863, all incorporated by reference in their entirety herein, and in particular Maxi-K channel blockers such as 1-(1-isobutyl-6-methoxy-1H-indazol-3-yl)-2-methylpropan-1-one; 1-[1-(2,2-dimethylpropyl)-6-methoxy-1H-indazol-3-yl]-2-methylpropan-1-one; 1-[1-(cyclohexylmethyl)-6-methoxy-1H-indazol-3-yl]-2-methylpropan-1-one; 1-(1-hexyl-6-methoxy-1H-indazol-3-yl)-2-methylpropan-1-one; 1-[1-(2-ethylhexyl)-6-methoxy-1H-indazol-3-yl]-2-methylpropan-1-one; 1-(3-isobutyryl-6-methoxy-1H-indazol-1-yl)buan-2-one; 1-(3-isobutyryl-6-methoxy-1H-indazol-1-yl)-3,3-dimethylbutan-2-one; 1-(3-cyclopentylcarbonyl)-6-methoxy-1H-indazol-1-yl)-3,3-dimethylbutan-2-one; 1-(3,3-dimethyl-2-oxobutyl)-6-methoxy-1H-indazole-3-carboxylic acid; and 1-[3-(3-hydroxypropanoyl)-6-methoxy-1H-indazol-1-yl]-3,3-dimethylbutan-2-one, a prostaglandin such as latanoprost, travaprost, unoprostone, rescula, S1033 (compounds set forth in U.S. Pat. Nos. 5,889,052; 5,296,504; 5,422,368; and 5,151,444); a hypotensive lipid such as lumigan and the compounds set forth in U.S. Pat. No. 5,352,708; a neuroprotectant disclosed in U.S. Pat. No. 4,690,931, particularly eliprodil and R-eliprodil as set forth in WO 94/13275, including memantine; and/or an agonist of 5-HT2 receptors as set forth in PCT/US00/31247, particularly 1-(2-aminopropyl)-3-methyl-1H-imdazol-6-ol fumarate and 2-(3-chloro-6-methoxy-indazol-1-yl)-1-methyl-ethylamine.

Use of the compounds of formula I for the manufacture of a medicament for treating ocular hypertension, glaucoma, macular edema, macular degeneration, for increasing retinal and optic nerve bead blood velocity, for increasing retinal and optic nerve oxygen tension, for providing a neuroprotective effect or for a combination thereof is also included in this invention.

The EP₄ agonist used in the instant invention can be administered in a therapeutically effective amount intravaneously, subcutaneously, topically, transdermally, parenterally or any other method known to those skilled in the art. Ophthalmic pharmaceutical compositions are preferably adapted for topical administration to the eye in the form of solutions, suspensions, ointments, creams or as a solid insert. Ophthalmic formulations of this compound may contain from 0.00001 to 5% and especially 0.00001 to 0.1% of medicament. Higher dosages as, for example, up to about 10% or lower dosages can be employed provided the dose is effective in reducing intraocular pressure, treating glaucoma, increasing blood flow velocity or oxygen tension. For a single dose, from between 0.0000001 to 0.05 mg, preferably 0.0000005 to 0.02 mg, and especially 0.000005 to 0.01 mg of the compound can be applied to the human eye.

The pharmaceutical preparation which contains the compound may be conveniently admixed with a non-toxic pharmaceutical organic carrier, or with a non-toxic pharmaceutical inorganic carrier. Typical of pharmaceutically acceptable carriers are, for example, water, mixtures of water and water-miscible solvents such as lower alkanols or aralkanols, vegetable oils, peanut oil, polyalkylene glycols, petroleum based jelly, ethyl cellulose, ethyl oleate, carboxymethyl-cellulose, polyvinylpyrrolidone, isopropyl myristate and other conventionally employed acceptable carriers. The pharmaceutical preparation may also contain non-toxic auxiliary substances such as emulsifying, preserving, wetting agents, bodying agents and the like, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, thimerosal, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetracetic acid, and the like. Additionally, suitable ophthalmic vehicles can be used as carrier media for the present purpose including conventional phosphate buffer vehicle systems, isotonic boric acid vehicles, isotonic sodium chloride vehicles, isotonic sodium borate vehicles and the like. The pharmaceutical preparation may also be in the form of a microparticle formulation. The pharmaceutical preparation may also be in the form of a solid insert. For example, one may use a solid water soluble polymer as the carrier for the medicament. The polymer used to form the insert may be any water soluble non-toxic polymer, for example, cellulose derivatives such as methylcellulose, sodium carboxymethyl cellulose, (hydroxyloweralkyl cellulose), hydroxyethyl cellulose, hydroxypropyl cellulose, hydroxypropylmethyl cellulose; acrylates such as polyacrylic acid salts, ethylacrylates, polyactylamides; natural products such as gelatin, alginates, pectins, tragacanth, karaya, chondrus, agar, acacia; the starch derivatives such as starch acetate, hydroxymethyl starch ethers, hydroxypropyl starch, as well as other synthetic derivatives such as polyvinyl alcohol, polyvinyl pyrrolidone, polyvinyl methyl ether, polyethylene oxide, neutralized carbopol and xanthan gum, gellan gum, and mixtures of said polymer.

Suitable subjects for the administration of the formulation of the present invention include primates, man and other animals, particularly man and domesticated animals such as cats, rabbits and dogs.

The pharmaceutical preparation may contain non-toxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid, and the like.

The ophthalmic solution or suspension may be administered as often as necessary to maintain an acceptable IOP level in the eye. It is contemplated that administration to the mammalian eye will be from once up to three times daily.

For topical ocular administration the novel formulations of this invention may take the form of solutions, gels, ointments, suspensions or solid inserts, formulated so that a unit dosage comprises a therapeutically effective amount of the active component or some multiple thereof in the case of a combination therapy.

The compounds of the instant invention are also useful for mediating the bone modeling and remodeling processes of the osteoblasts and osteoclasts. See PCT US99/23757 filed Oct. 12, 1999 and incorporated herein by reference in its entirety. The major prostaglandin receptor in bone is EP₄, which is believed to provide its effect by signaling via cyclic AMP. See Ikeda T, Miyaura C, Ichikawa A, Narumiya S, Yoshiki S and Suda T 1995, In situ localization of three subtypes (EP₁, EP₃ and EP₄) of prostaglandin E receptors in embryonic and newborn mice., J Bone Miner Res 10 (sup 1): S172, which is incorporated by reference herein in its entirety. Use of the compounds of formula I for the manufacture of a medicament for mediating the bone modeling and remodeling processes are also included in this invention.

Thus, another object of the present invention is to provide methods for stimulating bone formation, i.e. osteogenesis, in a mammal comprising administering to a mammal in need thereof a therapeutically effective amount of an EP₄ receptor subtype agonist of formula I.

Still another object of the present invention to provide methods for stimulating bone formation in a mammal in need thereof comprising administering to said mammal a therapeutically effective amount of an EP₄ receptor subtype agonist of formula I and a bisphosphonate active. Use of the compounds of formula I for the manufacture of a medicament for stimulating bone formation is also included in this invention.

Yet another object of the present invention to provide pharmaceutical compositions comprising a therapeutically effective amount of an EP₄ receptor subtype agonist of formula I and a bisphosphonate active.

It is another object of the present invention to provide methods for treating or reducing the risk of contracting a disease state or condition related to abnormal bone resorption in a mammal in need of such treatment or prevention, comprising administering to said mammal a therapeutically effective amount of an EP₄ receptor subtype agonist of formula I. Use of the compounds of formula I for the manufacture of a medicament for treating or reducing the risk of contracting a disease state or condition related to abnormal bone resorption is also included in this invention.

The disease states or conditions related to abnormal bone resorption include, but are not limited to, osteoporosis, glucocorticoid induced osteoporosis, Paget's disease, abnormally increased bone turnover, periodontal disease, tooth loss, bone fractures, rheumatoid arthritis, periprosthetic osteolysis, osteogenesis imperfecta, metastatic bone disease, hypercalcemia of malignancy, and multiple myeloma.

Within the method comprising administering a therapeutically effective amount of an EP₄ receptor subtype agonist of formula I and a bisphosphonate active, both concurrent and sequential administration of the EP₄ receptor subtype agonist of formula I and the bisphosphonate active are deemed within the scope of the present invention. Generally, the formulations are prepared containing 5 or 10 mg of a bisphosphonate active, on a bisphosphonic acid active basis. With sequential administration, the agonist and the bisphosphonate can be administered in either order. In a subclass of sequential administration the agonist and bisphosphonate are typically administered within the same 24 hour period. In yet a further subclass, the agonist and bisphosphonate are typically administered within about 4 hours of each other.

A non-limiting class of bisphosphonate actives useful in the instant invention are selected from the group consisting of alendronate, cimadronate, clodronate, tiludronate, etidronate, ibandronate, neridronate, olpandronate, risedronate, piridronate, pamidronate, zolendronate, pharmaceutically acceptable salts thereof, and mixtures thereof.

A non-limiting subclass of the above-mentioned class in the instant case is selected from the group consisting of alendronate, pharmaceutically acceptable salts thereof, and mixtures thereof.

A non-limiting example of the subclass is alendronate monosodium trihydrate.

In the present invention, as it relates to bone stimulation, the agonist is typically administered for a sufficient period of time until the desired therapeutic effect is achieved. The term “until the desired therapeutic effect is achieved”, as used herein, means that the therapeutic agent or agents are continuously administered, according to the dosing schedule chosen, up to the time that the clinical or medical effect sought for the disease or condition being mediated is observed by the clinician or researcher. For methods of treatment of the present invention, the compounds are continuously administered until the desired change in bone mass or structure is observed. In such instances, achieving an increase in bone mass or a replacement of abnormal bone structure with normal bone structure are the desired objectives. For methods of reducing the risk of a disease state or condition, the compounds are continuously administered for as long as necessary to prevent the undesired condition. In such instances, maintenance of bone mass density is often the objective.

Nonlimiting examples of administration periods can range from about 2 weeks to the remaining lifespan of the mammal. For humans, administration periods can range from about 2 weeks to the remaining lifespan of the human, preferably from about 2 weeks to about 20 years, more preferably from about 1 month to about 20 years, more preferably from about 6 months to about 10 years, and most preferably from about 1 year to about 10 years.

The instant compounds are also useful in combination with known agents useful for treating or preventing bone loss, bone fractures, osteoporosis, glucocorticoid induced osteoporosis, Paget's disease, abnormally increased bone turnover, periodontal disease, tooth loss, osteoarthritis, rheumatoid arthritis, periprosthetic osteolysis, osteogenesis imperfecta, metastatic bone disease, hypercalcemia of malignancy, and multiple myeloma. Combinations of the presently disclosed compounds with other agents useful in treating or preventing osteoporosis or other bone disorders are within the scope of the invention. A person of ordinary skill in the art would be able to discern which combinations of agents would be useful based on the particular characteristics of the drugs and the disease involved. Such agents include the following: an organic bisphosphonate; a cathepsin K inhibitor; an estrogen or an estrogen receptor modulator; an androgen receptor modulator; an inhibitor of osteoclast proton ATPase; an inhibitor of HMG-CoA reductase; an integrin receptor antagonist; an osteoblast anabolic agent, such as PTH; calcitonin; Vitamin D or a synthetic Vitamin D analogue; and the pharmaceutically acceptable salts and mixtures thereof. A preferred combination is a compound of the present invention and an organic bisphosphonate. Another preferred combination is a compound of the present invention and an estrogen receptor modulator. Another preferred combination is a compound of the present invention and an estrogen. Another preferred combination is a compound of the present invention and an androgen receptor modulator. Another preferred combination is a compound of the present invention and an osteoblast anabolic agent.

Regarding treatment of abnormal bone resorption and ocular disorders, the formula I agonists generally have an EC₅₀ value from about 0.001 nM to about 100 microM, although agonists with activities outside this range can be useful depending upon the dosage and route of administration. In a subclass of the present invention, the agonists have an EC₅₀ value of from about 0.0001 microM to about 10 microM. In a further subclass of the present invention, the agonists have an EC₅₀ value of from about 0.0001 microM to about 1 microM. EC₅₀ is a common measure of agonist activity well known to those of ordinary skill in the art and is defined as the concentration or dose of an agonist that is needed to produce half, i.e. 50%, of the maximal effect. See also, Goodman and Gilman's, The Pharmacologic Basis of Therapeutics, 9th edition, 1996, chapter 2, E. M. Ross, Pharmacodynamics, Mechanisms of Drug Action and the Relationship Between Drug Concentration and Effect, and PCT US99/23757, filed Oct. 12, 1999, which are incoroporated by reference herein in their entirety.

The herein examples illustrate but do not limit the claimed invention. Each of the claimed compounds are EP₄ agonists and are useful for a number of physiological ocular and bone disorders.

The compounds of this invention can be made, with some modification, in accordance with U.S. Pat. No. 6,043,275, EP0855389, WO 03/047417 (U.S. Ser. No. 60/337,228), WO 03/047513 (U.S. Ser. No. 60/338,117), U.S. Ser. No. 60/406,530 (Merck Docket No. MC060), WO 2004/085430 and WO 01/46140, all of which are incorporated herein by reference in their entirety. The following non-limiting schemes and examples given by way of illustration is demonstrative of the present invention.

The preparation of compounds from the current invention can be accomplished according to general schemes 1 and 2, and is further illustrated in the experimental section.

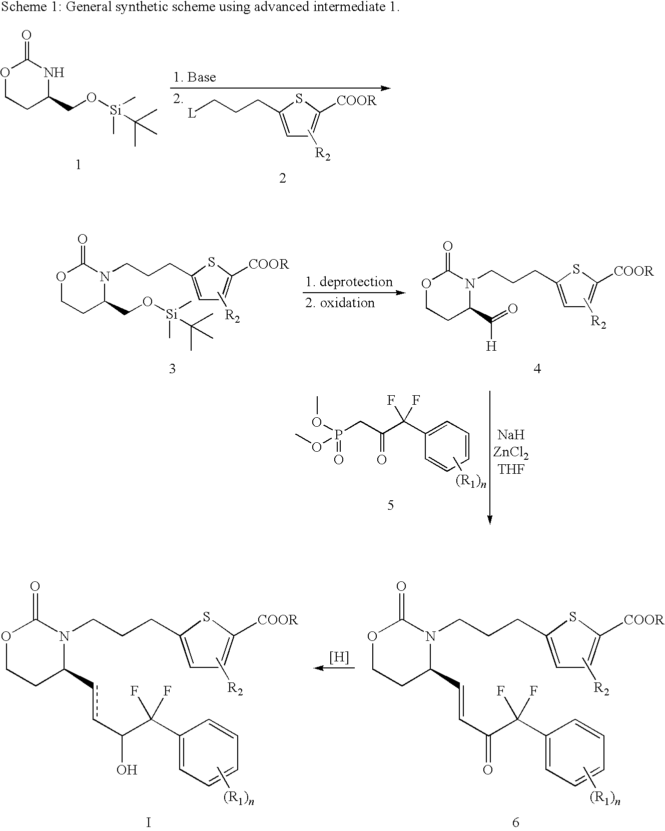

(4R)-4-({[tert-butyl(dimethyl)silyl]oxy}methyl)-1,3-oxazinan-2-one (1) (WO 2004/085430) was first treated with a strong base such as potassium hexamethyldisilazide (KHMDS) or NaH followed by treatment with reagent 2 (L=I, MeSO₂O or Br, etc.) in either THF (tetrahydrofuran) or DMF (dimethylformamide) to give the alkylation product 3. Deprotection of the t-butyldimethylsilyl (TBS) group with 1N aqueous HCl (hydrochloride) or with TBAF (tetrabutylammonium fluoride) followed by oxidation of the resultant alcohol with a suitable oxidant gave aldehyde 4. 4 was then reacted with reagent 5 using NaH as the base and ZnCl₂ as the Lewis acid to give ketone 6. Reduction of 6 with suitable reducing reagents afforded I.

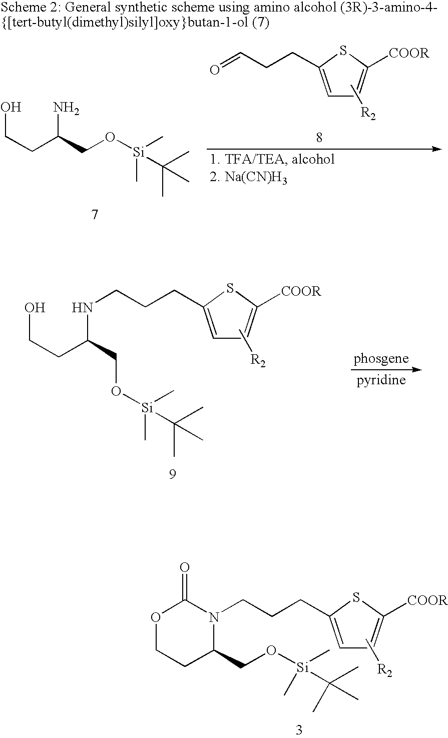

Reductive amination of amino alcohol 7 (WO 2004/085430) with a suitable aldehyde 8 followed by cyclization with phosgene using pyridine as the base gave 1,3-oxazinane intermediate 3. The intermediate can be processed to the desired product according to Scheme 1.

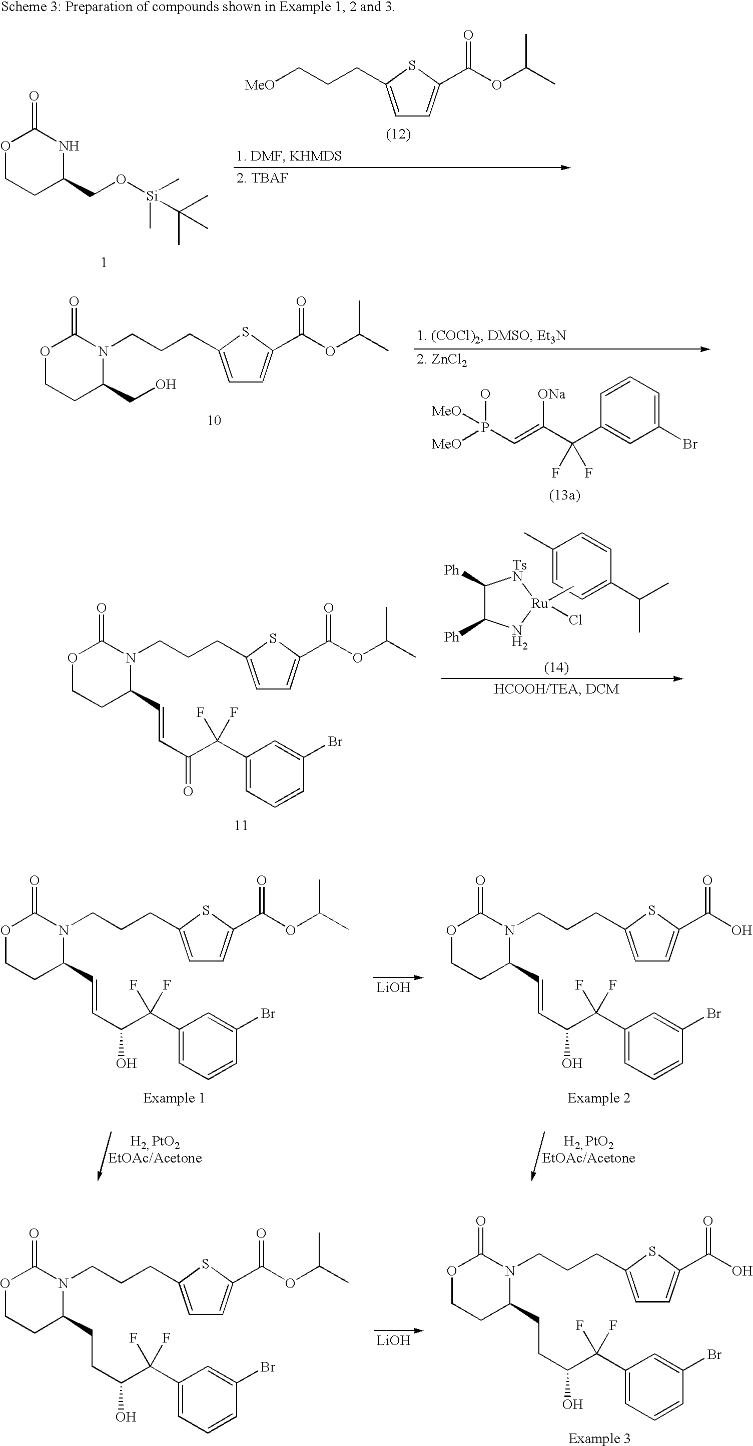

### Step 1

### Isopropyl 5-{3-[(4R)-4-({[tert-butyl(dimethyl)silyl]oxy}methyl)-2-oxo-1,3-oxazinan-3-yl]propyl}thiophene-2-carboxylate

To a solution of (4R)-4-({[tert-butyl(dimethyl)silyl]oxy}methyl)-1,3-oxazinan-2-one (1) (WO 2004/085430) (11 g, 44.8 mmol) in TBF (400 mL) at rt (room temperature) under N₂ was added KHMDS (99 mL, 0.5M in toluene) dropwise (gel like ppt formation) and the mixture was stirred at rt for 0.5 h (hour). Mesylate 12 (20 g, 65.3 mmol) in THF (150 mL) was then added via a cannula and the mixture was heated to 60° C. overnight and cooled to rt. The mixture was quenched with saturated NH₄Cl and then extracted with ethyl acetate. The crude product was purified by flash (10-50% ethyl acetate/hexanes) to give a yellow oil.

### Step 2

### Isopropyl 5-{3-[(4R)-4-(hydroxymethyl)-2-oxo-1,3-oxazinan-3-yl]propyl}thiophene-2-carboxylate

To a solution of alkylation product from above (20 g, 43.9 mmol) in THF (200 mL) was added HCl (50 mL, 1N) and the mixture was stirred at rt for 4 h. The volatiles were removed in vacuo and the residue purified by flash (30-50% acetone/toluene or 50-100% EA (ethyl acetate)/hexanes) to give the desired product 10 as a light yellow solid. ¹H NMR (Acetone-d₆): δ7.64 (1H, d, J=3.7 Hz), 7.00 (1 H, d, J=3.7 Hz), 4.39-4.31 (1H, m), 4.16-4.12 (2H, m), 3.83 (3H's), 3.72-3.70 (2H, m), 3.60-3.50 (2 H, m), 3.29-3.21 (1H, m), 2.92 (2H, t, J=7.7 Hz), 2.15-1.99 (4H, m).

### Step 3

### Isopropyl 5-{3-[(4R)-4-formyl-2-oxo-1,3-oxazinan-3-yl]propyl}thiophene-2-carboxylate

To a solution of DMSO (dimethyl sulfoxide) in DCM (dichloromethane) at −78° C. was added oxalyl chloride (141 uL, 1.615 mmol, 1.1 eq) dropwise and the mixture was stirred for 30 min. To this solution was added alcohol 10 (460 mg, 1.468 mmol) in DCM (8 mL) via a cannula and the mixture stirred for 15 min followed by the addition of triethylamine (495 uL, 3.52 mmol, 2.4 eq). The mixture was stirred at −78° C. for 30 min (minutes) and allowed to warm to rt. The DCM was evaporated in vacuo and the residue resuspended in EtOAc and filtered. The solid was washed with EtOAc thoroughly and the filtrate and washing solutions were concentrated to give the desired product. ¹H NMR (ppm)(CDCl₃): 9.65 (1H, s), 7.64 (1H, d, J=3.7 Hz), 6.84 (1H, d, J=3.6 Hz), 4.34-4.22 (1H, m), 4.09-4.01 (2H, m), 3.87 (3H, s), 3.82-3.74 (1H, m), 3.13-3.03 (1H, m), 2.94-2.86 (2H, m), 2.31-2.19 (2H, m), 2.07-1.99 (2 H, m). This reaction works equally well with sulfurtrioxide/pyridine complex.

### Step 4

### Isopropyl 5-(3-{(4R)-4-[(1E)-4-(3-bromophenyl)-4,4-difluoro-3-oxobut-1-en-1-yl]-2-oxo-1,3-oxazinan-3-yl}propyl)thiophene-2-carboxylate (11)

To a solution of aldehyde isopropyl 5-{3-[(4R)-4-formyl-2-oxo-1,3-oxazinan-3-yl]propyl}thiophene-2-carboxylate from above (265 mg, 0.802 mmol) in THF (3 ml) was added zinc chloride 0.5M THF (1.9 mL, 0.936 mmol, 1.1 eq) followed by reagent 13a (sodium salt of dimethyl [3-(3-bromophenyl)-3,3-difluoro-2-oxopropyl]phosphonate) (420 mg, 1.106 mmol, 1.3 eq) as a solid and the mixture was heated to 70° C. overnight and cooled to rt. The cloudy solution was diluted with 1N HCl/water and extracted with ethyl acetate (EA) (2×). The EA layers were washed with water and dried over MgSO₄. The crude was purified by flash (50-80% EA/hexane) to give desired product 11 which was contaminated with the phosphonate reagent by NMR. ¹H NMR (ppm) (Acetone-d₆): 7.81 (2H, s), 7.75-7.53 (2H, m), 7.20-7.12 (1H, m), 6.89-6.81 (1H, m), 4.99-4.91 (1H, m), 4.46 (1H, m), 3.80-3.72 (3H, m), 3.61-3.49 (1H, m), 2.84-2.76 (1H, m), 2.36-2.22 (3H, m), 1.60-1.48 (4H, m), 1.37-1.23 (4H, m), 1.20 (6H, d, J=6.3 Hz).

### Step 5

### Preparation of Catalyst 14

The catalyst was prepared by mixing 1 mol equiv of [RuCl₂(p-cymene)₂], 2 mol equiv (R,R)-N-Tosyl-1,2-diphenylethylene-1,2-diamine and 4.2 mol equiv of Et₃N in iPrOH at 80° C. for 1 h. After solvent removal, the solid was washed with cold H₂O and the recrystallized from MeOH to give the catalyst as an orange solid.

The catalyst could also be generated in situ by mixing 0.02 mol equiv of [RuCl₂(p-cymene)₂] and 0.04 mol equiv of the (R,R)—N-Tosyl-1,2-diphenylethylene-1,2-diamine in DCM (dichloromethane) in the presence of 0.04 mol equiv of 1M solution KOtBu in THF. After aging for 10 min at RT(room temperature), Et₃N was added followed by HCO₂H and a solution of the enone in DCM.

### EXAMPLE 1

### Isopropyl 5-(3-{(4R)-4-[(1E,3R)-4-(3-bromophenyl)-4,4-difluoro-3-hydroxybut-1-en-1-yl]-2-oxo-1,3-oxazinan-3-yl}propyl)thiophene-2-carboxylate

To a solution of ketone 11 (500 mg, 0.877 mmol) in DCM (5 ml) was added formic acid (52 μl, 1.31 mmol) and triethylamine (148 μl, 1.05 mmol) and the mixture was stirred at rt for 10 min. To it was then added Ru catalyst 14 (28 mg, 0.044 mmol) and the mixture was stirred at rt until all starting material was consumed by TLC analysis. The mixture was diluted with EA and washed with 1N HCl. The organic layer was dried, filtered and concentrated in vacuo and the crude was purified by flash chromatography (25-75% EA/hexanes) to give the desired product in a diastereomeric ratio of 10-40/1 in favor of the desired isomer. The diastereomeric mixture was further purified by chiral AD (30% i-PrOH/exanes) to give the title compound. ¹H NMR δ (ppm)(Acetone-d₆): 7.69 (2H, d, J=6.4 Hz), 7.62 (1H, d, J=3.7 Hz), 7.55 (1H, d, J=7.8 Hz), 7.45 (1H, t, J=8.2 Hz), 6.97 (1H, d, J=3.7 Hz), 5.86 (1 H, dd, J=6.7, 15.5 Hz), 5.74 (1H, dd, J=5.5, 15.5 Hz), 5.23 (1H, d, J=5.8 Hz), 5.18-5.10 (1H, m), 4.76-4.68 (1H, m), 4.19-4.04 (3H, m), 3.65-3.57 (1H, m), 2.90-2.84 (1H, m), 2.22-2.14 (1H, m), 2.02-1.94 (2H, m), 1.84-1.78 (1H, m), 1.32 (6H, d, J=6.7 Hz). MS (+APCI): 572.0, 574.0.

### EXAMPLE 2

### 5-(3-{(4R)-4-[(1E,3R)-4-(3-bromophenyl)-4,4-difluoro-3-hydroxybut-1-en-1-yl]-2-oxo-1,3-oxazinan-3-yl}propyl)thiophene-2-carboxylic Acid

The isopropyl ester in Example I was treated with LiOH in Methanol/water to give the corresponding acid. ¹H NMR (ppm)(Acetone-d₆): δ7.69 (2H, d, J=7.0 Hz), 7.64 (1H, d, J=3.7 Hz), 7.55 (1H, d, J=7.7 Hz), 7.45 (1H, t, J=8.1 Hz), 6.98 (1H, d, J=3.6 Hz), 5.91-5.83 (1H, m), 5.76-5.68 (1H, m), 5.23 (1H, s), 4.75-4.69 (1H, m), 4.19-4.04 (4H, m), 3.66-3.58 (1H, m), 2.91-2.83 (3H, m), 2.22-2.14 (1H, m), 2.03-1.95 (3H, m), 1.86-1.78 (1H, m); MS (−ESI): m/z 527.9, 529.9.

### EXAMPLE 3

### 5-3-{(4S)-4-[(3R)-4-(3-bromophenyl)-4,4-difluoro-3-hydroxybutyl]-2-oxo-1,3-oxazinan-3-yl}propyl)thiophene-2-carboxylic Acid

To a solution of Example 1 (110 mg, 0.2 mmol) in EtOAc (10 mL) and acetone (10 mL) was added PtO₂ (5 mg). The resulting black reaction mixture was subjected to H₂ (1 atm (atmosphere)) for 18 h. The solution was filtered over a pad of celite and the organic solvent was removed in vacuo. The crude product was purified by flash column chromatography to afford the ester which was hydrolyzed in the usual manner to the title compound. MS (+ESI) m/z 532.2, 534.2.

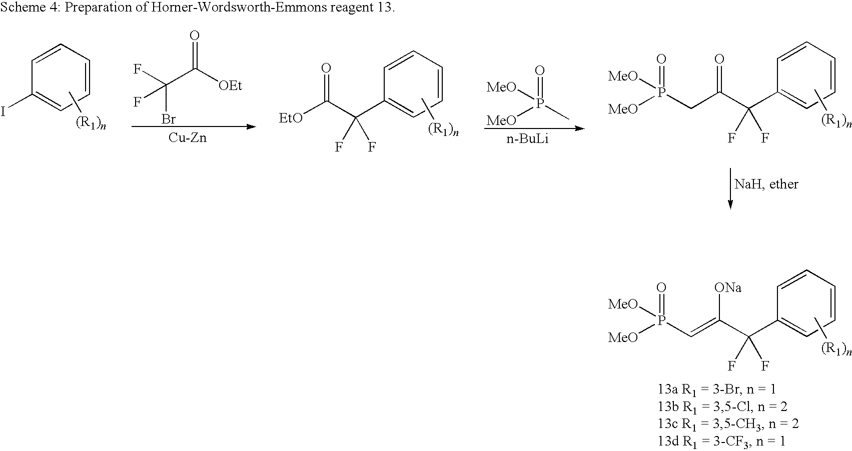

### Preparation of Reagent 13a

Step 1: To a solution of 3-bromo-iodobenzene (14.1 g, 50 mmol) and ethyl bromo-α,α-difluoroacetate (10.1 g, 50 mmol) in DMSO (40 mL) was added copper bronze (7 g, 110 mmol) and the suspension was heated to 55° C. for 2.5 days and cooled to rt. The mixture was quenched with KH₂PO₄ and filtered. The solid was washed with EA/water and the filtrate was separated. The aqueous layer was extracted with ether (2×) and the organic phases were combined, washed with water and brine. The crude was purified by flash (5-10% EA/hex) to give 10.7 g of the desired product as a colorless oil. To a solution of dimethyl methylphosphonate (4.1 g, 33 mmol) in THF (100 mL) at −78° C. was added n-BuLi (12.6 mL, 2.5M (minutes) in hexanes) dropwise and the mixture was stirred at −78° C. for 1 h. To this solution was then added ethyl α,α-difluoro-3-bromophenylacetate (8.37 g, 30 mmol) in THF via a cannula and the mixture was stirred at −78° C. for 2 h and quenched with 2.2 mL AcOH and water. After warming to rt, the mixture was extracted with EA (3×). The organic layers were washed with brine, dried over sodium sulfate, filtered and concentrated in vacuo to give the desired product dimethyl [3-3-bromophenyl)-3,3-difluoro-2-oxopropyl]phosphonate as a colorless oil. To a solution of this oil (8.28 g, 23.19 mmol) in ether at rt was added sodium hydride 60% (974 mg, 24.35 mmol, 1.05 eq) portionwise and the white suspension stirred at rt for 1 h. The mixture was filtered and the white solid washed with ether/hexane. The solid thus obtained was dried under high vacuum to give 13a (white powder). Reagents 13b-13d were prepared in a similar manner.

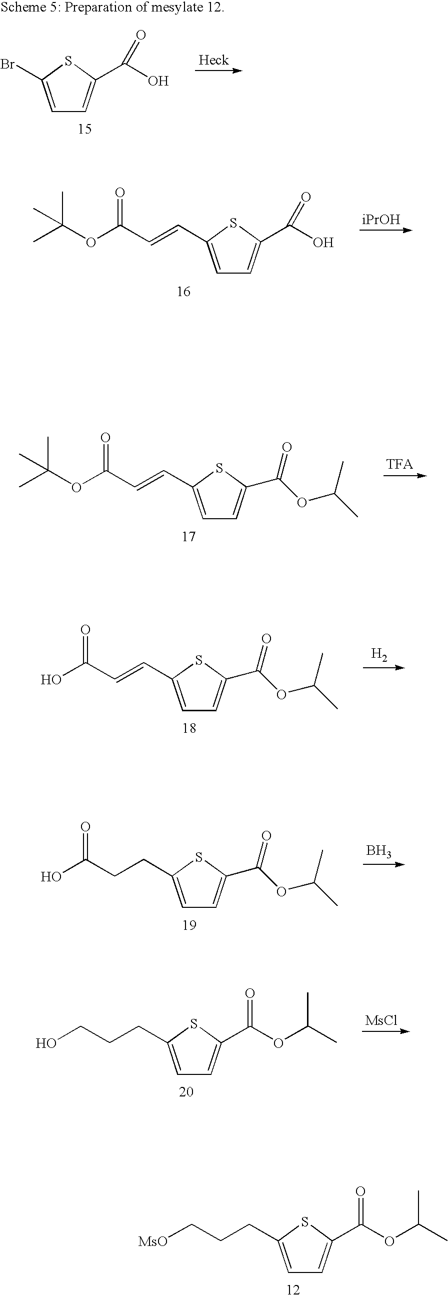

### Step 1

### 5-[(1E)-3-tert-butoxy-3-oxoprop-1-en-1-yl]thiophene-2-carboxylic Acid (16)

To a degassed solution of PPh₃ (3.2 g, 12.2 mmol), NEt₃ (171 mL, 1218 mmol), t-butyl acrylate (118 mL, 812 mmol), and acid 15 (42 g, 203 mmol) in 100 mL CH₃CN was added Pd(OAc)₂ (911 mg, 4.1 mmol) and the solution was heated to reflux for 12 hours. The solution was cooled to rt and dilute HCl was added and the product was extracted with CH₂Cl₂. The organic layer was dried over Na₂SO₄, filtered and concentrated to yield the crude product which immediately solidified. The resultant solid was collected and washed with hexanes to yield 16.

### Step 2

### Isopropyl 5-[(1E)-3-tert-butoxy-3-oxoprop-1-en-1-yl]thiophene-2-carboxylate (17)

A solution of acid 16 (44 g, 173 mmol) and N,N-Dimethylformamide diisopropyl acetal (60 g, 342 mmol) in 500 mL of 1,2-Dichloroethane was heated to reflux for 12 hours. The solution was cooled to rt. and concentrated to yield the crude product which was purified by column chromotography to yield 17.

### Step 3

### (2E)-3-[5-isopropoxycarbonyl)-2-thienyl]acrylic Acid (18)

To a solution of ester 17 (45 g, 152 mmol) in 500 mL of dichloromethane was added 45 mL of TFA (trifluoroacetic acid) slowly and the solution was stirred at rt. for 12 hours. To the solution was added 300 mL of PhMe and followed by concentration in vacuo. The resultant solid was collected and washed with hexanes to yield 18.

### Step 4

### 3-[5-(isopropoxycarbonyl)-2-thienyl]propanoic Acid (19)

To a solution of acid 18 (45 g, 152 mmol) in 200 mL of isopropanol was added 5.32 g of 10% Pd on carbon and the mixture was subjected to 40 psi of hydrogen for 12 hours. The solution was filtered over celite followed by concentration in vacuo. The resultant solid was collected and washed with hexanes to yield 19.

### Step 5

### Isopropyl 5-(3-hydroxypropyl)thiophene-2-carboxylate (20)

To a solution of acid 19 (17.3 g, 71.4 mmol) in 200 mL THF at −10 C was added borane dimethyl sulfide complex (71.5 mL of a 2M solution in THF, 143 mmol) slowly. The solution was stirred for 3 hours followed by slow quenching with 10 mL of MeOH. The solution was stirred for another 2 hours and concentrated. Further addition of 100 mL of MeOH followed by concentration was repeated twice to yield the crude alcohol which was purified by column chromotography to yield 20.

### Step 6

### Mesylate Isopropyl 5-{3-[(methylsulfonyl)oxy]propyl}thiophene-2-carboxylate (12)

To a solution of alcohol 20 (15 g, 65.7 mmol) in 200 mL dichloromethane at −20° C. was added triethylamine (18.4 mL, 131 mmol) followed by mesyl chloride (6.2 mL, 79 mmol) slowly. The solution was stirred for 3 hours followed by quenching water. The product was extracted with CH₂Cl₂ and the organic layer was dried over Na₂SO₄, filtered and concentrated to yield the crude product. The crude mesylate was purified by column chromatography to yield 12.

### EXAMPLE 4

### 5-(3-{(4R)-4-[(1E)-4,4-difluoro-3-hydroxy-4-phenylbut-1-en-1-yl]-2-oxo-1,3-oxazinan-3-yl}propyl)thiophene-2-carboxylic Acid

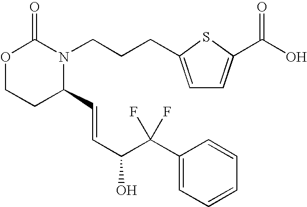

This compound was prepared from intermediate 10 and dimethyl (3-phenyl-3,3-difluoro-2-oxopropyl)phosphonate (WO 2004/085430) according to Scheme 3. ¹H NMR δ(ppm)(Acetone-d₆): 7.64 (1H, d, J=3.7 Hz), 7.55-7.51 (2H, m), 7.47 (3H, d, J=6.1 Hz), 6.98 (1H, d, J=3.7 Hz), 5.83 (1 H, dd, J=6.2, 15.5 Hz), 5.73-5.67 (1H, m), 5.10 (1H, s), 4.72-4.66 (1H, m), 4.16-4.04 (4H, m), 3.65-3.55 (1H, m), 2.90-2.82 (1H, m), 2.20-2.12 (1H, m), 1.97 (2H, t, J=7.5 Hz), 1.82-1.76 (1H, m). MS (−APCI): m/z 450.3 (M−1)⁻.

### EXAMPLE 5

### Isopropyl 5-(3-{(4R)-4-[(1E,3R)-4-(3,5-dichlorophenyl)-4,4-difluoro-3-hydroxybut-1-en-1-yl]-2-oxo-1,3-oxazinan-3-yl}propyl)thiophene-2-carboxylate

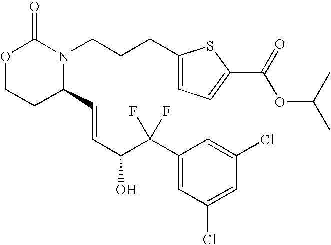

¹H NMR δ(ppm)(Acetone-d₆): 7.63-7.61 (2H, m), 7.52 (2H, s), 6.96 (1H, d, J=3.2 Hz), 5.89 (1H, dd, J=15.5 Hz, J=6.6 Hz), 5.78 (1H, dd, J=15.4 Hz, J=5.0 Hz), 5.37 (1H, d, J=5.8 Hz), 5.17-5.11 (1H, m), 4.79-4.71 (1H, m), 4.15-4.19 (3H, m), 3.67-3.60 (1H, m), 2.87-2.95 (3H, m), 2.24-2.15 (1H, m), 2.02-1.80 (2H, m), 1.82 (1H, dd, J=14.0 Hz, J=3.3 Hz), 1.32 (9, d, J=6.2 Hz).

### EXAMPLE 6

### 5-(3-{(4R)-4-[(1E,3R)-4-3,5-dichlorophenyl)-4,4-difluoro-3-hydroxybut-1-en-1-yl]-2-oxo-1,3-oxazinan-3-yl}propyl)thiophene-2-carboxylic Acid

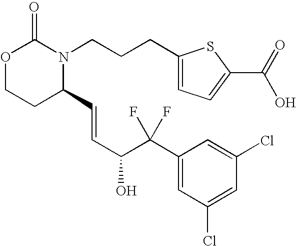

The isopropyl ester from above was treated with LiOH in Methanol/water to give the corresponding acid MS (+ESI): m/z 520.2 (M+1)⁺.

### EXAMPLE 7

### Isopropyl 5-3-{(4S)-4-[(3R)-4-3,5-dichlorophenyl)-4,4-difluoro-3-hydroxybutyl]-2-oxo-1,3-oxazinan-3-yl}propyl)thiophene-2-carboxylate

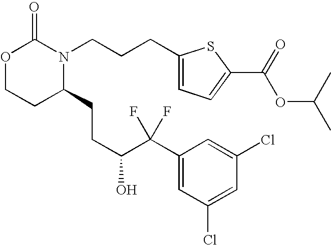

The double bond in Example 4 was processed to the title compound by reduction with PtO₂ in ethyl acetate/acetone under hydrogen (1 atm) to give the saturated compound as discussed in Scheme 1. ¹H NMR δ(ppm)(Acetone-d₆): 7.66 (1H, s), 7.61 (1H, d, J=3.7 Hz), 7.54 (2H, d, 1.7 Hz), 6.98 (1H, d, J=3.7 Hz), 5.17-5.12 (m, 1), 4.95 (1, d, J=6.7 Hz), 4.27 (1H, dt, J=11.1 Hz, J=3.0 Hz), 4.17-4.06 (2H, m), 3.64-3.55 (2H, m), 3.18-3.12 (1H, m), 2.90 (2H, t, J=7.8 Hz), 2.12-1.91 (5H, m), 1.87-1.80 (1H, m), 1.75-1.69 (1H, m), 1.50-1.42 (1H, m), 13.3 (6H, d, J=6.3 Hz).

### EXAMPLE 8

### 5-(3-{(4S)-4-[(3R)-4-(3,5-dichlorophenyl)-4,4-difluoro-3-hydroxybutyl]-2-oxo-1,3-oxazinan-3-yl}propyl)thiophene-2-carboxylic Acid

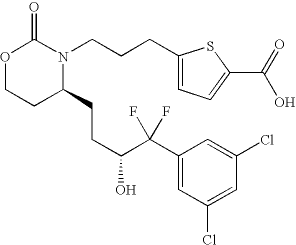

The isopropyl ester from above was treated with LiOH in Methanol/water to give the corresponding acid. ¹H NMR δ(ppm)(Acetone-d₆): 7.65 (1H, s), 7.63 (1H, d, 3.7 Hz), 7.54 (2H, d, J=1.6 Hz), 6.98 (1H, d, J=3.4 Hz), 4.27 (1H, dt, J=11.0 Hz, 2.9 Hz), 4.17-4.11 (2H, m), 3.64-3.51 (2H, m); 3.19-3.13 (1H, m), 2.90 (2H, t, J=7.8 Hz), 2.12-1.91 (3H, m), 1.87-1.81 (1H, m), 1.80-1.69 (1H, m), 1.50-1.42 (1H, m), 1.39-1.31 (1H, m).

### EXAMPLE 9

### Isopropyl 5-(3-{(4R)-4-[(1E,3R)-4-(3,5-dimethylphenyl)-4,4-difluoro-3-hydroxybut-1-en-1-yl]-2-oxo-1,3-oxazinan-3-yl}propyl)thiophene-2-carboxylate

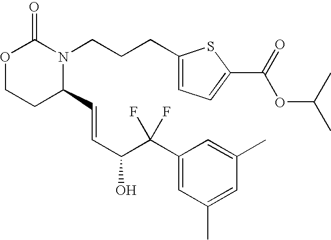

This compound was prepared from intermediate 10 and reagent 13c according to Scheme 3. ¹H NMR δ(ppm)(CDCl₃): 7.61 (1H, d, J=3.7 Hz), 7.07 (3H, s), 6.81 (1H, d, J=3.6 Hz), 5.74 (1H, dd, J=15.7 Hz, 5.9 Hz), 5.64 (1H, dd, J=15.6 Hz, 5.0 Hz), 5.22-5.16 (1H, m), 4.59-4.53 (1H, m), 4.15-4.10 (2H, m), 3.97-3.96 (1H, m), 3.69-3.61 (1H, m), 2.90-2.80 (3H, m), 2.35 (6H, s), 2.19-2.08 (1H, m), 2.00-1.93 (2H, m), 1.79-1.75 (1H, m), 1.35 (6H, d, J=6.2 Hz).

### EXAMPLE 10

### 5-(3-{(4R)-4-[(1E,3R)-4-3,5-dimethylphenyl)-4,4-difluoro-3-hydroxybut-1-en-1-yl]-2-oxo-1,3-oxazinan-3-yl}propyl)thiophene-2-carboxylic Acid

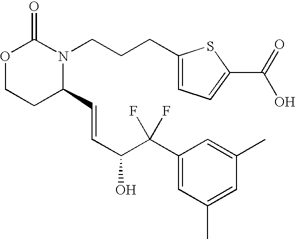

The isopropyl ester from above was treated with LiOH in Methanol/water to give the corresponding acid. ¹H NMR δ(ppm)(CDCl₃): 7.70 (1H, d, J=3.3 Hz), 7.07 (3H, s), 6.86 (1H, d, J=2.9 Hz), 5.75 (1H, dd, J=15.6 Hz, 5.9 Hz), 5.66 (1H, dd, J=15.6 Hz, 5.0 Hz), 4.60-4.54 (1H, m), 4.15-4.13 (2H, m), 4.00-3.95 (1H, m), 3.70-3.63 (1H, m), 2.89-2.81 (3H, m), 2.35 (6H, s), 2.22-1.94 (3H, m), 1.80-1.77 (1H, m).

### EXAMPLE 11

### 5-(3-{(4S)-4-[(3R)-4-(3,5-dimethylphenyl) 4,4-fluoro-3-hydroxybutyl]-2-oxo-1,3-oxazinan-3-yl}propyl)thiophene-2-carboxylic Acid

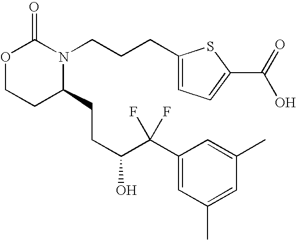

The acid from above was treated with PtO₂ in ethyl acetate/acetone under hydrogen (1 atm) to give corresponding acid. MS (−ESI): 480.2 (M−1)⁻.

### EXAMPLE 12

### Isopropyl 5-[3-((4R)-4-{(1E,3R)-4,4-difluoro-3-hydroxy-4-[3-(trifluoromethyl)phenyl]but 1-en-1-yl}-2-oxo-1,3-oxazinan-3-yl)propyl]thiophene-2-carboxylate

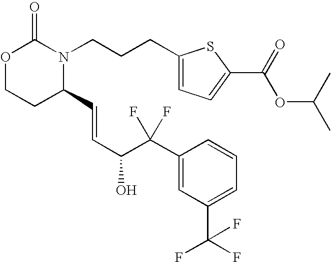

The compound was prepared from intermediate 10 and reagent 13d as described in Scheme 3. ¹H NMR δ(ppm)(Acetone-d₆): 7.88-7.84 (3H, m), 7.77-7.73 (1H, m), 7.61 (1H, d, J=3.7 Hz), 6.97 (1H, d, J=3.7 Hz), 5.88 (1H, dd, J=15.6 Hz, 6.6 Hz), 5.77 (1H, dd, J=15.4 Hz, 5.4 Hz), 5.27 (1H, d, J=5.8 Hz), 5.19-5.09 (1H, m), 4.81-4.74 (1H, m), 4.20-4.12 (3H, m), 3.65-3.58 (1H, m), 2.93-2.85 (3H, m), 2.22-2.13 (1H, m), 2.08-1.21 (2H, m), 1.83-1.76 (1H, m), 1.32 (6H, d, J=6.2 Hz).

### EXAMPLE 13

### 5-[3-(4R)-4-{(1E,3R)-4,4-difluoro-3-hydroxy-4-[3-(trifluoromethyl)phenyl]but-1-en-1-yl}-2-oxo-1,3-oxazinan-3-yl)propyl]thiophene-2-carboxylic Acid

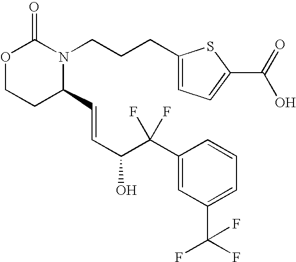

The isopropyl ester from above was treated with LiOH in Methanol/water to give the corresponding acid. MS (−ESI): m/z 518.5 (M-H)⁻.

### EXAMPLE 14

### Isopropyl 5-[3-((4S)-4-{(3R)-4,4-difluoro-3-hydroxy-4-[3-(trifluoromethyl)phenyl]butyl}-2-oxo-1,3-oxazinan-3-yl)propyl]thiophene-2-carboxylate

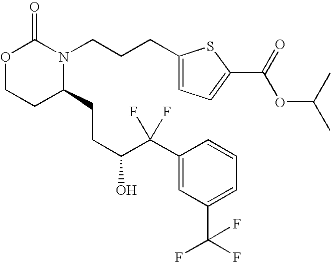

The above intermediate was processed to the title compound by reduction with PtO₂ in ethyl acetate/acetone under hydrogen (1 atm) as discussed in Scheme 3.

¹H NMR δ(ppm)(Acetone-d₆) 7.90-7.85 (3H, m), 7.80-7.73 (1H, m), 7.61 (1H, d, J=3.7 Hz), 6.97 (1H, d, J=3.7 Hz), 5.17-5.11 (1H, m), 4.95 (1H, d, 7.3 Hz), 4.31-4.23 (1H, m), 4.18-4.10 (2H, m), 3.65-3.51 (2H, m), 3.18-3.12 (1H, m), 2.89-2.86 (2H, t, J=8.0 Hz), 2.14-1.68 (7H, m), 1.51-1.40 (1H, m), 1.33 (6H, d, J=6.3 Hz).

### EXAMPLE 15

### 5-[3-((4S)-4-{(3R)-4,4-difluoro-3-hydroxy-4-[3-(trifluoromethyl)phenyl]butyl}-2-oxo-1,3-oxazinan-3-yl)propyl]thiophene-2-carboxylic Acid

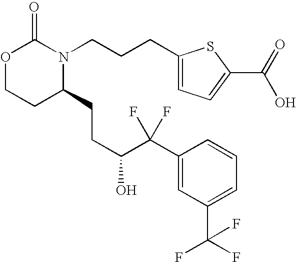

The isopropyl ester from above was treated with LiOH in Methanol/water to give the corresponding acid. MS (−ESI): m/z 520.0 (M-H)⁻.

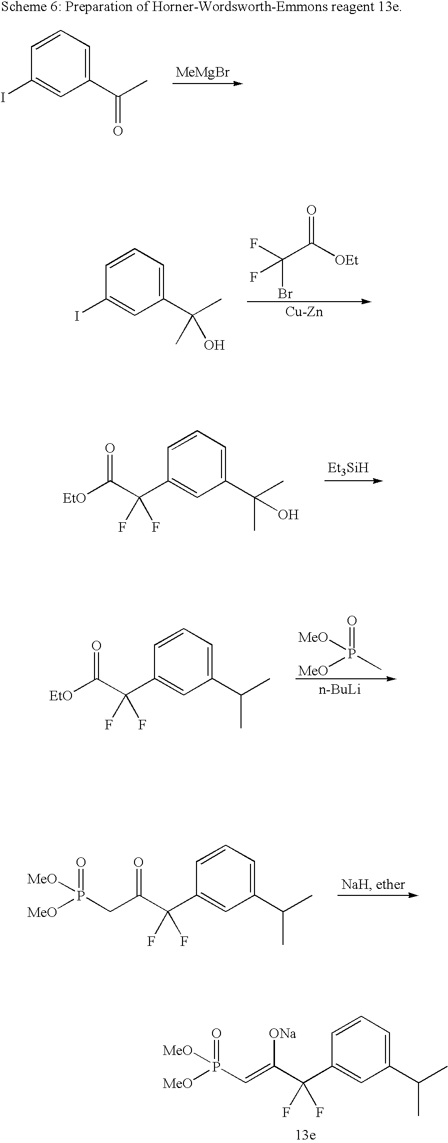

### Step 1

### 2-(3-iodophenyl)propan-2-ol

To a solution of 3-iodo-acetophenone (15 g, 61 mmol) in 100 μL THF at −78° C. was slowly added MeMgBr (60 mL of a 1.4M solution in THF, 84 mmol) and the solution was stirred for 1 hour as it warmed to 0° C. The reaction was quenched with saturated NH₄Cl and the crude product was extracted with ether (2×) and the organic phases were dried over Na₂SO₄, filtered and concentrated in vacuo. The crude was purified by flash (5-10% EA/hex) to give the desired product.

### Step 2

### Ethyl difluoro[3-(1-hydroxy-1-methylethyl)phenyl]acetate

To a solution of the 2-(3-iodophenyl)propan-2-ol (10 g, 38.2 mmol) in 50 mL DMSO was added ethyl bromo-α,α-difluoroacetate (7.75 g, 38.2 mmol) and copper bronze (5.34 g, 84 mmol) and the suspension was heated to 55° C. for 2.5 days and cooled to rt. The mixture was quenched with KH₂PO₄ and filtered. The solid was washed with EA/water and the filtrated was separated. The aqueous layer was extracted with ether (2×) and the organic phases were combined, washed with water and brine and then dried over Na₂SO₄, filtered and concentrated in vacuo. The crude was purified by flash (5-10% EA/hex) to give the desired product.

### Step 3

### Ethyl α,α-difluoro(3-isopropylphenyl)acetate

To a solution of the tertiary alcohol ethyl difluoro[3-(1-hydroxy-1-methylethyl)phenyl]acetate (7 g, 27.1 mmol) in 50 mL dichloromethane at −78° C. was added triethylsilane (21.7 mL, 136 mmol) followed by borontrifluoride diethyl etherate (16.7 mL, 136 nmol) and the solution stirred for 30 minutes. The mixture was quenched with solid NaHCO₃ and warmed to rt. Water was added an the aqueous layer was extracted with ether (2×) and the organic phases were combined, washed with water and brine and then dried over Na₂SO₄, filtered and concentrated in vacuo. The crude was purified by flash (5-10% EA/hex) to give the desired product.

### Step 4

### Dimethyl [3,3-difluoro-3-(3-isopropylphenyl)-2-oxopropyl]phosphonate

To a solution of dimethyl methylphosphonate (3.1 g, 25 mmol) in THF (100 mL) at −78° C. was added n-BuLi (14.2 mL of a 1.6M solution in hexanes, 22.7 mmol) dropwise and the mixture was stirred at the temperature for 1 h. To this solution was then added ethyl α,α-difluoro(3-isopropylphenyl)acetate (5.5 g, 22.7 mmol) in THF via a cannula and the mixture was stirred at −78° C. for 2 h and quenched with 2.2 mL AcOH and water. After warming to rt, the mixture was extracted with ethyl acetate (3×). The organic layers were washed with brine, dried over sodium sulfate, filtered and concentrated in vacuo to give the desired product as a colorless oil.

### Step 5

### Preparation of 13e

To a solution of this oil (3 g, 9.4 mmol) in ether/hexanes (1:1, 40 mL) at 0° C. was added sodium hydride 60% (375 mg, 9.37 mmol) portionwise and the white suspension was stirred at rt for 1 h. The mixture was filtered and the white solid washed with ether/hexanes. The solid thus obtained was dried under high vacuum to give 13e (white powder).

### EXAMPLE 16

### 5-(3-{(4R)-4-[(1E,3R)-4,4-difluoro-3-hydroxy-4-(3-isopropylphenyl)but-1-en-1-yl]-2-oxo-1,3-oxazinan-3-yl}propyl)thiophene-2-carboxylic Acid

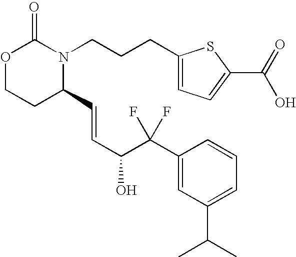

The title compound was prepared according to Scheme 3 using intermediate 10 and reagent 13e. MS (+ESI): m/z 494.0 (M+1)⁺.

### EXAMPLE 17

### 5-(3-{(4S)-4-[(3R)-4,4-difluoro-3-hydroxy-4-3-isopropylphenyl)butyl]-2-oxo-1,3-oxazinan-3-yl}propyl)thiophene-2-carboxylic Acid

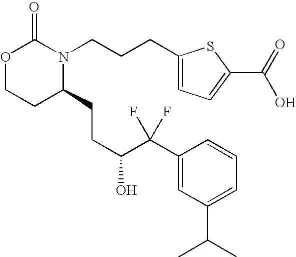

The title compound was prepared by hydrogenation of using PtO₂ as the catalyst. MS (+ESI): m/z 496.1 (M+1)⁺.

The preparation of compounds shown Examples 18 and 19 are shown in Scheme 7.

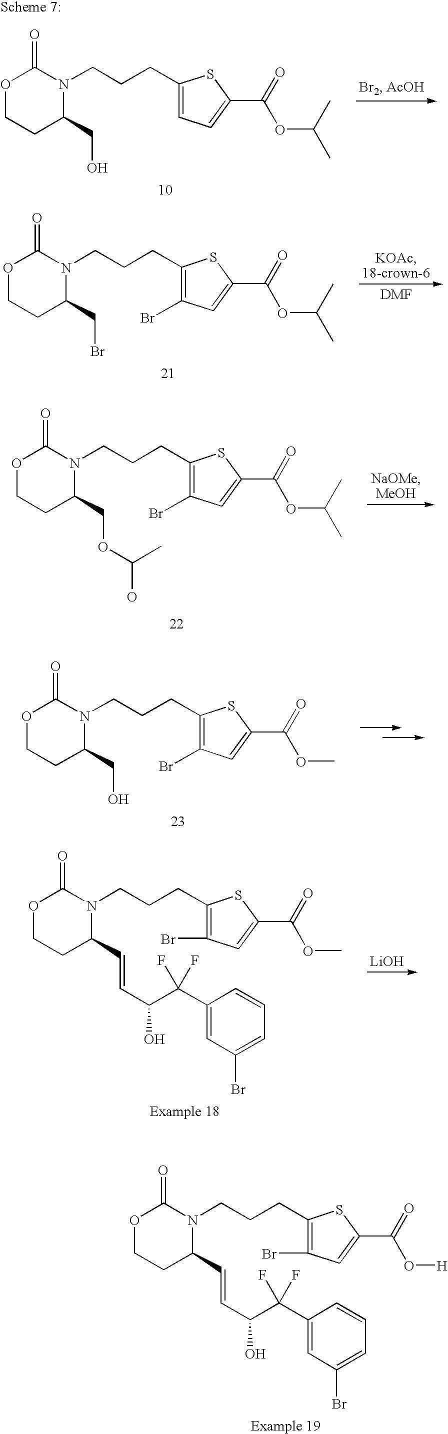

### Step 1

### Isopropyl 4-bromo-5-{3-[(4R)-4-bromomethyl)-2-oxo-1,3-oxazinan-3-yl]propyl}thiophene-2-carboxylate (21)

To a solution of thiophene 10 (0.700 g, 2.05 mmol) in AcOH (2 mL) was added bromine (0.140 mL, 2.67 mmol) dropwise. The solution was stirred for 18 h. The reaction mixture was diluted with 10% Na₂S₂O₃ (aq) and the aqueous layer was extracted with CH₂Cl₂ (3×). The combined organic was dried over Na₂SO₄ and the organic solvent was evaporated in vacuo. The crude product was purified by flash column chromatography (100% EtOAc) to afford 21 as a yellow oil. ¹H NMR (ppm)(Acetone-d₆): 7.61 (1H, s), 5.20-5.11 (1H, m), 4.37 (1H, dt, J=11.1 Hz, 3.1 Hz), 4.29-4.18 (3H, m), 3.82-3.77 (1H, m), 3.68-3.61 (1H, m), 3.33-3.27 (1H, m), 2.89 (2H, t, J=7.8 Hz), 2.25-2.16 (1H, m), 2.10-1.82 (3H, m), 1.34 (6H, d, J=6.3 Hz).

### Step 2

### Isopropyl 5-(3-{(4R)-4-[(acetyloxy)methyl]-2-oxo-1,3-oxazinan-3-yl}propyl)-4-bromothiophene-2-carboxylate (22)

To a stirred suspension of 18-crown-6 (2.10 g) and potassium acetate (1.19 g, 12.1 mmol) in dry DMF (75 mL) at 105° C. was added a solution of bromide 21 (0.782 g, 1.62 mmol) in DMF (15 mL). The reaction mixture was heated at 105° C. for 18 h. The reaction was cooled to room temperature and was diluted with H₂O. The aqueous layer was extracted with 1:1 EtOAc/ether (3×) and the combined organic layer was washed with brine and dried over Na₂SO₄. The organic solvent was concentrated in vacuo. The crude product was purified by flash column chromatography (100% EtOAc) to afford 22 as a yellow oil. MS (+ESI): m/z 462.1, 464.1.

### Step 3

### Isopropyl 4-bromo-5-{3-[(4R)-4-(hydroxymethyl)-2-oxo-1,3-oxazinan-3-yl]propyl}thiophene-2-carboxylate (23)

To a solution of acetate 22 (0.729 g, 1.58 mmol) in dry MeOH (10 mL) was added NaOMe (340 mg, 6.31 mmol) as a solid. After stirring for 2 h, the reaction was quenched with saturated NH₄Cl (aq) and the aqueous layer was extracted with EtOAc (3×). The combined organic layer was dried over Na₂SO₄ and concentrated. The crude product was purified by flash column chromatography (100% EtOAc) to afford 23 as a yellow oil. MS (+ESI): m/z 392.2, 394.2.

### EXAMPLE 18

### Methyl 4-bromo-5-3-{(4R)-4-[(1E,3R)-4-(3-bromophenyl)-4,4-difluoro-3-hydroxybut-1-en-1-yl]-2-oxo-1,3-oxazinan-3-yl}propyl)thiophene-2-carboxylate

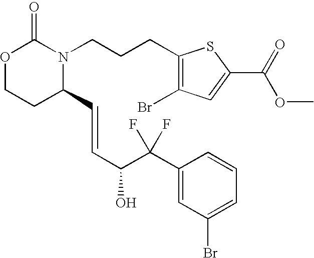

The above intermediate 23 was processed to the title compound as discussed. MS (+ESI): m/z 622.1, 624.1, 626.1.

### EXAMPLE 19

### 4-bromo-5-(3-{(4R)-4-[(1E,3R)-4-(3-bromophenyl)-4,4-difluoro-3-hydroxybut-1-en-1-yl]-2-oxo-1,3-oxazinan-3-yl}propyl)thiophene-2-carboxylic Acid

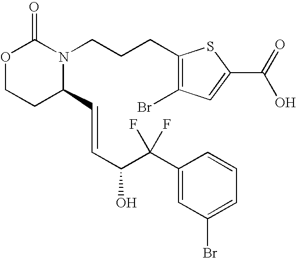

The methyl ester from above was treated with LiOH in Methanol/water to give the corresponding acid. MS (+ESI): m/z 610.2, 612.2.

### EXAMPLE 20

### 5-(3-{(4R)-4-[(1E,3R)-4-(3-bromophenyl)-4,4-difluoro-3-hydroxybut-1-en-1-yl]-2-oxo-1,3-oxazinan-3-yl}propyl)-3-fluorothiophene-2-carboxylic Acid

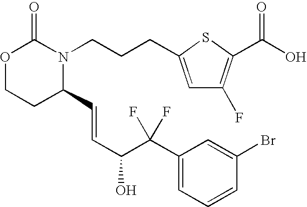

This compound was prepared according to Scheme 3 and 4. ¹H NMR δ (ppm)(Acetone): 7.70 (2H, m), 7.56 (1H, d, J=7.8 Hz), 7.49-7.43 (1H, m), 6.90 (1H, s), 5.88 (1H, dd, J=6.6, 15.6 Hz), 5.76 (1H, dd, J=5.5, 15.5 Hz), 5.23 (1H, s), 4.74 (1H, s), 4.20-4.13 (3H, m), 3.65-3.57 (1H, m), 2.93-2.81 (3H, m), 2.23-2.16 (1H, m), 2.01-1.95 (2H, m), 1.85-1.79 (1H, m).

### EXAMPLE 21

### 5-(3-{(4S-4-[(3R)-4-(3-bromophenyl)-4,4-difluoro-3-hydroxybutyl]-2-oxo-1,3-oxazinan-3-yl}propyl)-3-fluorothiophene-2 carboxylic Acid

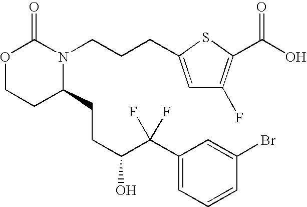

MS (−ESI):m/z 550.1, 548.1.

### EXAMPLE 22

### 5-(3-{(4S)-4-[(3R)-3-(4′-chloro-2′-methylbiphenyl-3-yl)-3-hydroxypropyl]-2-oxo-1,3-oxazinan-3-yl}propyl)thiophene-2-carboxylic Acid

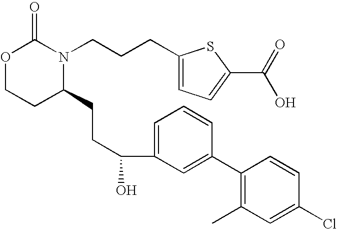

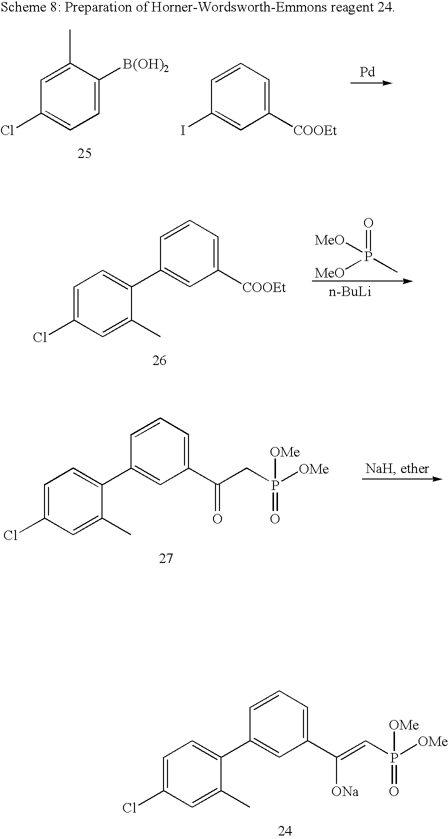

To a nitrogen purged solution of boronic acid 25 (8 g, 47 mmol), 3-iodo-ethylbenzoate (7 g, 25.4 mmol) in 100 mL dimethoxyethane at rt was added tetrakis(triphenylphosphine)palladium(0) (1 g, 0.865 mmol). To the solution was then added sodium carbonate (47 mL of a 1M solution in water, 47 mmol) and the resultant solution was heated to 85° C. for 3 hours. The reaction was cooled to rt and water was added. The crude product was extracted with ethyl acetate (2×) and the organic phases were dried over Na₂SO₄, filtered and concentrated in vacuo. The crude was purified by flash chromatography (5-10% ethyl acetate/hexanes) to give desired product 26.

To a solution of dimethyl methylphosphonate (3.73 mL, 35 mmol) in THF (100 mL) at −78° C. was added LHMDS (76 mL of a 1M solutin in THF, 76 mmol) dropwise and the mixture was stirred −78° C. for 1 h. To this solution was then added the ethyl ester 26 (8 g, 29.1 mmol) in THF via a cannula and the mixture was stirred for 2 h as it warmed to 0° C. and quenched with 1N HCl (80 mL). After warming to rt, the mixture was extracted with ethyl acetate (3×). The organic layers were washed with brine, dried over sodium sulfate, filtered and concentrated in vacuo to give the crude product which was purified by flash chromatography (5-10% ethyl acetate/hexanes) to yield of the desired product 27.

To a solution of the phosphonate 27 (8.96 g, 25.4 mmol) in ether/hexanes (1:1, 200 mL) at 0° C. was added sodium hydride 60% (1040 mg, 26 mmol) portionwise and the white suspension stirred at rt for 1 h. The mixture was filtered and the white solid washed with ether/hexanes. The solid obtained was dried under high vacuum to yield the desired sodium salt 24 as a pale yellow powder.

The title compound was prepared from alcohol 10 and reagent 24 using procedures described in Scheme 3. MS (ESI) m/z 528.1 (M)⁺.

### EXAMPLE 23

### Isopropyl 5-(3-{(4R)-4-[(1E,3R)-4-(3-bromo-5-iodophenyl)-4,4-difluoro-3-hydroxybut-1-en-1-yl]-2-oxo-1,3-oxazinan-3-yl}propyl)thiophene-2-carboxylate

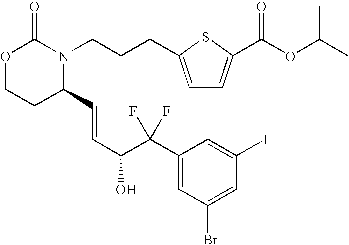

MS (ESI) m/z 697.9 (M+1)⁺, 700.1 (M+2)⁺.

### I. Effects of an EP4 Agonist on Intraocular Pressure (IOP) in Rabbits and Monkeys.

### Animals

Drug-naïve, male Dutch Belted rabbits and female cynomolgus monkeys are used in this study. Animal care and treatment in this investigation are in compliance with guidelines by the National Institute of Health (NIH) and the Association for Research in Vision and Opthalmology (ARVO) resolution in the use of animals for research. All experimental procedures str approved by the Institutional Animal Care and Use Committee of Merck and Company.

### Drug Preparation and Administration

Drug concentrations are expressed in terms of the active ingredient (base). The compounds of this invention are dissolved in a suitable ophthalmic solution (e.g., 0.5% polysorbate-80, 0.1% EDTA, 0.02% benzalkonium chloride, 4.5% mannitol in 5 mM citrate buffer at pH 5-7) at 0.00063, 0.00013% for rabbit studies and 0.0063, 0.0019%, 0.00063 and 0.000063% for monkey studies. Drug or vehicle aliquots (25 ul) are administered topically unilaterally or bilaterally. In unilateral applications, the contralateral eyes receive an equal volume of vehicle (0.5% polysorbate-80, 0.1% EDTA, 0.02% benzalkonium chloride, 4.5% mannitol in 5 mM citrate buffer at pH 5-7). Proparacaine (0.5%) is applied to the cornea prior to tonometry to minimize discomfort. Intraocular pressure (IOP) is recorded using a pneumatic tonometer (Alcon Applanation Pneumatonograph) or equivalent.

### Analysis

The results are expressed as the changes in IOP from the basal level measured just prior to administration of drug or vehicle and represent the mean, plus or minus standard deviation. Statistical comparisons are made using the Student's t-test for non-paired data between responses of drug-treated and vehicle-treated animals and for paired data between ipsilateral and contralateral eyes at comparable time intervals. The significance of the date is also determined as the difference from the “t-0” value using Dunnett's “t” test. Asterisks represent a significance level of p<0.05.

### A. Intraocular Pressure Measurement in Rabbits

Male Dutch Belted rabbits weighing 2.5-4.0 kg are maintained on a 12-hour light/dark cycle and rabbit chow. All experiments are performed at the same time of day to minimize variability related to diurnal rhythm. IOP is measured before treatment then the compounds of this invention or vehicle are instilled (one drop of 25 ul) into one or both eyes and IOP is measured at 30, 60, 120, 180, 240, 300, and 360 minutes after instillation. In some cases, equal number of animals treated bilaterally with vehicle only are evaluated and compared to drug treated animals as parallel controls.

### B. Intraocular Pressure Measurements in Monkeys.

Unilateral ocular hypertension of the right eye is induced in female cynomolgus monkeys weighing between 2 and 3 kg by photocoagulation of the trabecular meshwork with an argon laser system (Coherent NOVUS 2000, Palo Alto, USA) using the method of Lee at al. (1985). The prolonged increase in intraocular pressure (IOP) results in changes to the optic nerve head that are similar to those found in glaucoma patients.

For IOP measurements, the monkeys are kept in a sitting position in restraint chairs for the duration of the experiment. Animals are lightly anesthetized by the intramuscular injection of ketamine hydrochloride (3-5 mg/kg) approximately five minutes before each IOP measurement and one drop of 0.5% proparacaine was instilled prior to recording IOP. IOP is measured using a pneumatic tonometer (Alcon Applanation Tonometer) or a Digilab pneumatonometer (Bio-Rad Ophthalmic Division, Cambridge, Mass., USA).

IOP is measured before treatment and generally at 30, 60, 124, 180, 300, and 360 minutes after treatment. Baseline values are also obtained at these time points generally two or three days prior to treatment. Treatment consists of instilling one drop of 25 ul of the compounds of this invention (0.000063 to 0.0063%) or vehicle. At least one-week washout period is employed before testing on the same animal. The normotensive (contralateral to the hypertensive) eye is treated in an exactly similar manner to the hypertensive eye. IOP measurements for both eyes are compared to the corresponding baseline values at the same time point. Results are expressed as mean plus-or-minus standard deviation in mm Hg. The activity range of the compounds of this invention for ocular use is between 0.01 and 100,000 nM.

Compounds from the current invention (i.e., Example 1) showed improved ocular tolerability in animal species such as rabbits and cynomolgus monkeys compared to compounds disclosed in WO 2004/085430 (i.e., Example 2). For example, in a vehicle panel-controlled study in Dutch-Belted rabbits for 5 days (topical, bilateral, b.i.d) with either 2.2 μM (25 μL) of an ophthalmic solution of Example 1 or vehicle (0.5% Polysorbate-80, 0.02% benzalkonium chloride, 0.1% EDTA, 4.5% mannitol in 5 mM citrate), no drug treatment related findings were noted for the present compound. In comparison, Example 2 of WO 2004/085430 demonstrated treatment related findings of very slight bilateral hyperemia, very slight conjunctival chemosis and slight ocular discharge at the same dose. Similarly a 5-fold improvement in eye closure was observed with the present compound in New Zealand White rabbits after ocular dosing. The present compound also demonstrated a 3-5-fold improvement in ocular adverse effects (conjuctival hyperemia, partial eye closure, etc.) in Cynomolgus Monkeys.

### II. Radioligand Binding Assays:

The assays used to test these compounds were performed essentially as described in: Abramovitz M, Adam M, Boie Y, Carriere M, Denis D, Godbout C, Lamontagne S, Rochette C, Sawyer N, Tremblay N M, Belley M, Gallant M, Dufresne C, Gareau Y, Ruel R, Juteau H, Labelle M, Ouimet N, Metters K M. The utilization of recombinant prostanoid receptors to determine the affinities and selectivities of prostaglandins and related analogs. Biochim Biophys Acta 2000 Jan. 17; 1483(2):285-293 and discussed below:

### Stable Expression of Prostanoid Receptors in the Human Embryonic Kidney (HEK) 293(EBNA) Cell Line

Prostanoid receptor (PG) cDNAs corresponding to full length coding sequences were subcloned into the appropriate sites of the mammalian expression vector pCEP4 (Invitrogen) pCEP4PG plasmid DNA was prepared using the Qiagen plasmid preparation kit (QIAGEN) and transfected into HEK 293(EBNA) cells using LipofectAMIE® (GIBCO-BRL) according to the manufacturers' instructions. HEK 293(EBNA) cells expressing the cDNA together with the hygromycin resistance gene were selected in Dulbecco's Modified Eagle Medium (DMEM) supplemented with 10% heat inactivated fetal bovine serum, 1 mM sodium pyruvate, 100 U/ml Penicillin-G, 100 μg/ml Streptomycin sulphate, 250 μg/ml active GENETICIN™ (G418) (all from Life Technologies, Inc./BRL) and 200 μg/ml bygromycin (Calbiochem). Individual colonies were isolated after 2-3 weeks of growth under selection using the cloning ring method and subsequently expanded into clonal cell lines. Expression of the receptor cDNA was assessed by receptor binding assays.

HEK 293(EBNA) cells were grown in supplemented DMEM complete medium at 37° C. in a humidified atmosphere of 6% CO₂ in air, then harvested and membranes prepared by differential centrifugation (1000×g for 10 min, then 160,000×g for 30 min, all at 4° C.) following lysis of the cells by nitrogen cavitation at 800 psi for 30 min on ice in the presence of protease inhibitors (2 mM phenylmethylsulfonylfluoride, 10 μM E-64, 100 μM leupeptin and 0.05 mg/ml pepstatin). The 160,000×g pellets were resuspended in 10 mM HEPES/KOH (pH 7.4) containing 1 mM EDTA at approximately 5-10 mg/ml protein by Dounce homogenisation (Dounce A; 10 strokes), frozen in liquid nitrogen and stored at −80° C.

### Prostanoid Receptor Binding Assays

Prostanoid receptor binding assays were performed in a final incubation volume of 0.2 ml in 10 mM MES/KOH (pH 6.0) (EP subtypes, FP and TP) or 10 mM HEPES/KOH (pH 7.4) (DP and IP), containing 1 mM EDTA, 10 mM MgCl₂ (EP subtypes) or 10 mM MnCl₂ (DP, FP, IP and TP) and radioligand [0.5-1.0 nM [³]HPGE₂ (181 Ci/mmol) for EP subtypes, 0.7 nM [³H]PGD2 (115 Ci/mmol) for DP, 0.95 nM [³H]PGF_{2□} (170 Ci/mmol) for FP, 5 nM [³H]iloprost (16 Ci/mmol) for IP and 1.8 nM [³H]SQ 29548 (46 Ci/mmol) for TP]. EP₃ assays also contained 100 μM GTP□S. The reaction was initiated by addition of membrane protein (approximately 30 μg for EP₁, 20 μg for EP₂, 2 μg for EP₃, 10 μg for EP₄, 60 μg for FP, 30 μg for DP, 10 μg for IP and 10 μg for TP) from the 160,000×g fraction. Ligands were added in dimethylsulfoxide (Me₂SO) which was kept constant at 1% (v/v) in all incubations. Non-specific binding was determined in the presence of 1 μM of the corresponding non-radioactive prostanoid. Incubations were conducted for 60 min (EP subtypes, FP and IP) or 30 min (DP and TP) at 30° C. (EP subtypes, DP, FP and TP) or room temperature (EP) and terminated by rapid filtration through a 96-well Unifilter GF/C (Can berra Packard) prewetted in assay incubation buffer without EDTA (at 4° C.) and using a Tomtec Mach III 96-well semi-automated cell harvester. The filters were washed with 3-4 ml of the same buffer, dried for 90 min at 55° C. and the residual radioactivity bound to the individual filters determined by scintillation counting with addition of 50 μl of Ultima Gold F (Canberra Packard) using a 1450 MicroBeta (Wallac). Specific binding was calculated by subtracting non-specific binding from total binding. Specific binding represented 90-95% of the total binding and was linear with respect to the concentrations of radioligand and protein used. Total binding represented 5-10% of the radioligand added to the incubation media.

The activity range of the compounds of this invention for bone use is between 0.01 and 100,000 nM.

### Bone Resorption Assays:

### 1. Animal Procedures:

For mRNA localization experiments, 5-week old Sprague-Dawley rats (Charles River) are euthanized by CO₂, their tibiae and calvariae are excised, cleaned of soft tissues and frozen immediately in liquid nitrogen. For EP₄ regulation experiments, 6-week old rats are given a single injection of either vehicle (7% ethanol in sterile water) or an anabolic dose of PGE₂ (Cayman Chemical, Ann Arbor, Mich.), 3-6 mg/kg in the same vehicle) intraperitoneally. Animals are euthanized at several time points post-injection and their tibiae and calvariae, as well as samples from lung and kidney tissues are frozen in liquid nitrogen.

### 2. Cell Cultures

RP-1 periosteal cells are spontaneously immortalized from primary cultures of periosteal cells from tibae of 4-week old Sprague-Dawley rats and are cultured in DMEM (BRL, Gaithersburg, Md.) with 10% fetal bovine serum (JRH Biosciences, Lenexa, Kans.). These cells do not express osteoblastic phenotypic markers in early culture, but upon confluence, express type I collagen, alkaline phosphatase and osteocalcin and produce mineralized extracellular matrix.

RCT-1 and RCT-3 are clonal cell lines immortalized by SV40 large T antigen from cells released from fetal rat calvair by a cmbination collagenase/hyaluronidase digestion. RCT-1 cells, derived from cells released during the first 10 minutes of digestion (fraction I), are cultured in RPMI 1640 medium (BRL) with 10% fetal bovine serum and 0.4 mg/ml G418 (BRL). These cells differentiate and express osteoblastic features upon retinoic acid treatment. RCT-3 cells, immortalized from osteoblast-enriched fraction III cells, are cultured in F-12 medium (BRL) with 5% Fetal bovine serum and 0.4 mg/ml G418. TRAB-11 cells are also immortalized by SV40 large T antigen from adult rat tibia and are cultured in RPMI 1640 medium with 10% FBS and 0.4 mg/ml G418. ROS 17/2.8 rat osteosarcoma cells are cultured in F-12 containing 5% FBS. Osteoblast-enriched (fraction III) primary fetal rat calvaria cells are obtained by collagenase/hyaluronidase digestion of calvariae of 19 day-old rat fetuses. See Rodan et al., Growth stimulation of rat calvaria osieoblastic cells by acidic FGF, Endocrinology, 121, 1919-1923 (1987), which is incorporated by reference herein in its entirety. Cells are released during 30-50 minutes digestion (fraction III) and are cultured in F-12 medium containing 5% FBS.

P815 (mouse mastocytoma) cells, cultured in Eagles MEM with 10% FBS, and NRK (normal rat kidney fibroblasts) cells, cultured in DMEM with 10% FBS, are used as positive and negative controls for the expression of EP₄, respectively. See Abramovitz et al., Human prostanoid receptors: cloning and characterization. In: Samulesson B. et al. ed) Advances in prostaglandin, Thrombosznes and leukotriene research, vol. 23, pp. 499-504 (1995) and de Larco et al., Epithelioid andfibroblastic rat kidney cell clones: EGF receptors and the effect of mouse sarcoma virus transformation, Cell Physiol., 94, 335-342 (1978), which are both incorporated by reference herein in their entirety.

### 3. Northern Blot Analysis:

Total RNA is extracted from the tibial metaphysis or diaphysis and calvaria using a guanidinium isothiocyanate-phenol-chloroform method after pulverizing frozen bone samples by a tissue homogenizer. See P. Chomczynski et al., Single-step method of RNA isolation by acid guanidium thiocyanate-phenol-chloroform extraction., Analyt Biochem, 162, 156-159 (1987), which is incorporated by reference herein in its entirety. RNA samples (20 mg) are separated on 0.9% agarose/formaldehyde gels and transferred onto nylon membranes (Boehringer Mannheim, Germany). Membranes are prehybridized in Hybrisol I (Oncor, Gaithersburg, Md.) and 0.5 mg/ml sonicated salmon sperm DNA (Boehringer) at 42° C. for 3 hours and are hybridized at 42° C. with rat EP₂ and mouse EP₄ cDNA probes labeled with [³²P]-dCTP (Amersham, Buckinghamshire, UK) by random priming using the rediprime kit (Amersham). After hybridization, membranes are washed 4 times in 2×SSC+0.1% SDS at room temperature for a total of 1 hour and once with 0.2×SSC+0.1% SDS at 55° C. for 1 hour and then exposed to Kodak XAR 2 film at −70° C. using intensifying screens. After developing the films, bound probes are removed twice with 0.1% SDS at 80° C. and membranes are hybridized with a human GAPDH (Glyceraldehyde 3-Phosphate Dehydrogenase) cDNA probe (purchased from Clontech, Palo Alto, Calif.) for loading control.

### 4. In-Situ Hybridization:

Frozen tibiae are sectioned coronally at 7 mm thickness and sections are mounted on charged slides (Probe On Plus, Fisher Scientific, Springfield, N.J.) and are kept at −70° C. until hybridization. cRNA probes are labeled with ³⁵S-UTPgS (ICN, Costa Mesa, Calif.) using a Riboprobe II kit (Promega Madison, Wis.). Hybridization is performed overnight at 50° C. See M. Weinreb et al., Different pattern of alkaline phosphalase, osteopontin and osteocalcin expression in developing rat bone visualized by in-situ hybridization, J. Bone Miner Res., 5, 83′-842 (1990) and D. Shinar et al., Expression of alphav and beta3 integrin subunits in rat osteoclasts in situ, J. Bone Miner. Res., 8, 403-414 (1993), which are both incorporated by reference herein in their entirety. Following hybridization and washing, sections are dipped in Ilford K5 emulsion diluted 2:1 with 6% glycerol in water at 42° C. and exposed in darkness at 4° C. for 12-14 days. Slides are developed in Kodak D-19 diluted 1:1 with water at 15°, fixed, washed in distilled water and mounted with glycerol-gelatin (Sigma) after hematoxylin staining. Stained sections are viewed under the microscope (Olympus, Hamburg, Germany), using either bright-field or dark-field optics.

### 5. Expression of EP₄ in Osteoblastic Cell Lines and in Bone Tissue.

The expression of EP₄ and EP₂ mRNA is examined in various bone derived cells including osteoblast-enriched primary rat calvaria cells, immortalized osteoblastic cell lines from fetal rat calvaria or from adult rat tibia and an osteoblastic osteosarcoma cell line. Most of the osteoblastic cells and cell lines show significant amounts of 3.8 kb EP₄ mRNA, except for the rat osteosarcoma cell line ROS 17/2.8. Consistent with this finding, in ROS 17/2.8 cells PGE₂ has no effect on intracellular cAMP, which is markedly induced in RCT-3 and TRAB-11 cells. Treatment of RCT-1 cells with retinoic acid, which promotes their differentiation, reduces the levels of EP₄ mRNA. NRK fibroblasts do not express EP₄ mRNA, while P815 mastocytoma cells, used as positive controls, express large amounts of EP₄ mRNA. In contrast to EP₄ mRNA, none of the osteoblastic cells and cell lines express detectable amounts of EP₂ mRNA in total RNA samples. Expression of EP₄ mRNA in osteoblastic cells, EP₄ is also expressed in total RNA isolated from tibiae and calvariae of 5-week-old rats. In contrast, no EP₂ mRNA is found in RNA from tibial shafts.

6. PGE₂ Induces the Expression of EP₁ mRNA in RP-1 Periosteal Cells and in Adult Rat Tibiae

PGE₂ enhances its own production via upregulation of cyclooxygenase 2 expression in osteoblasts and in bone tissue thus autoamplifying its own effects. PGE₂ also increases the levels of EP₄ mRNA. RP-1 cells are immortalized from a primary culture of adult rat tibia periosteum is examined. These cells express osteoblast phenotypic markers upon confluence and form mineralized bone matrix when implanted in nude mice. Similar to the other osteoblastic cells examined, RP-1 periosteal cells express a 3.8 kb EP4 transcript. Treatment with PGE₂ (10⁻⁶ M) rapidly increases EP₄ mRNA levels peaking at 2 hours after treatment. PGE₂ has no effect on EP₄ mRNA levels in the more differentiated RCT-3 cells pointing to cell-type specific regulation of EP₄ expression by PGE₂. EP₂ in RNA is not expressed in RP-1 cells before or after treatment with PGE₂.

To examine if PGE₂ regulates EP₄ mRNA levels in vivo in bone tissue, five-week-old male rats are injected with PGE₂ (3-6 mg/Kg). Systemic administration of PGE₂ rapidly increased EP₄ mRNA levels in the tibial diaphysis peaking at 2 h after injection. A similar effect of PGE₂ on EP₄ mRNA is observed in the tibial metaphysis and in calvaria. PGE₂ induces EP₄ mRNA levels in vitro in osteogenic periosteal cells and in vivo in bone tissue in a cell type-specific and tissue-specific manner. PGE₂ does not induce EP₂ mRNA in RP-1 cells nor in bone tissue.

7. Localization of EP₄ mRNA Expression in Bone Tissue

In situ hybridization is used in order to localize cells expressing EP₄ in bone. In control experiment (vehicle-injected) rats, low expression of EP₄ is detected in bone marrow cells. Administration of a single anabolic dose of PGE₂ increased the expression of EP₄ in bone marrow cells. The distribution of silver grains over the bone marrow is not uniform and occurs in clumps or patches in many areas of the metaphysis. Within the tibial metaphysis, EP₄ expression is restricted to the secondary spongiosa area and is not seen in the primary spongiosa. Hybridization of similar sections with a sense probe (negative control) does not show any signal.

EP₄ is expressed in osteoblastic cells in vitro and in bone marrow cells in vivo, and is upregulated by its ligand, PGE₂.

### 8. Agonists of the Present Invention

Using standard methods for measuring agonist activity, the following compounds are evaluated in cell cultures and in EP₄ receptor cell-free systems to determine the agonist activity of the compounds in terms of their EC₅₀ value.

What is claimed is:

1. A compound having the structural formula I: 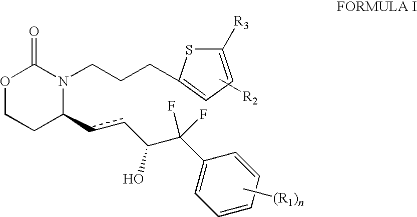
or a pharmaceutically acceptable salt, enantiomer, diastereomer, prodrug or mixture thereof, wherein,
R and R₄ independently represent H, or C₁₋₆ alkyl;
R₁ independently represents hydrogen, C₁₋₆ alkyl, halogen, CF₃, aryl, said aryl optionally substituted with 1 to 3 groups of halogen, C₁₋₆ alkyl, CF₃, or N(R₄)₂;
R₂ represents H, or halogen;
R₃ represents COOR or carboxylic acid isotere;
n represents 0-3; and
- - - represents a double or single bond.

2. A compound according to claim 1 wherein R₃ is COOR or tetrazole.

3. A compound according to claim 2 wherein R₃ is COOR.

4. A compound according to claim 3 wherein R is H, isopropyl, ethyl or, methyl.

5. A compound according to claim 1 wherein R₁ is halogen, C₁₋₆ alkyl or CF₃.

6. A compound according to claim 3 wherein R is H or isopropyl, R₁ is bromine and R₂ is hydrogen.

7. A compound according to claim 5 wherein n is 0, 1 or 2.

8. A compound which is:
Isopropyl 5-(3-{(4R)-4-[(1E,3R)-4-(3-bromophenyl)-4,4-difluoro-3-hydroxybut-1-en-1-yl]-2-oxo-1,3-oxazinan-3-yl}propyl)thiophene-2-carboxylate;
Isopropyl 5-(3-{(4R)-4-[(1E)-4-(3-bromophenyl)-4,4-difluoro-3-oxobut-1-en-1-yl]-2-oxo-1,3-oxazinan-3-yl}propyl)thiophene-2-carboxylate;
Isopropyl 5-(3-{(4R)-4-[(1E)-4-(3-bromophenyl)-4,4-difluoro-3-hydroxybut-1-en-1-yl]-2-oxo-1,3-oxazinan-3-yl}propyl)thiophene-2-carboxylate;
5-(3-{(4R)-4-[(1E,3R)-4-(3-bromophenyl)-4,4-difluoro-3-hydroxybut-1-en-1-yl]-2-oxo-1,3-oxazinan-3-yl}propyl)thiophene-2-carboxylic acid;
Isopropyl 5-(3-{(4S)-4-[(3R)-4-(3-bromophenyl)-4,4-difluoro-3-hydroxybutyl]-2-oxo-1,3-oxazinan-3-yl}propyl)thiophene-2-carboxylate;
5-(3-{(4S)-4-[(3R)-4-(3-bromophenyl)-4,4-difluoro-3-hydroxybutyl]-2-oxo-1,3-oxazinan-3-yl}propyl)thiophene-2-carboxylic acid;
5-(3-{(4R)-4-[(1E)-4,4-difluoro-3-hydroxy-4-phenylbut-1-en-1-yl]-2-oxo-1,3-oxazinan-3-yl}propyl)thiophene-2-carboxylic acid;
Isopropyl 5-(3-{(4R)-4-[(1E,3R)-4-(3,5-dichlorophenyl)-4,4-difluoro-3-hydroxybut-1-en-1-yl]-2-oxo-1,3-oxazinan-3-yl}propyl)thiophene-2-carboxylate;
5-(3-{(4R)-4-[(1E,3R)-4-(3,5-dichlorophenyl)-4,4-difluoro-3-hydroxybut-1-en-1-yl]-2-oxo-1,3-oxazinan-3-yl}propyl)thiophene-2-carboxylic acid;
Isopropyl 5-(3-{(4S)-4-[(3R)-4-(3,5-dichlorophenyl)-4,4-difluoro-3-hydroxybutyl]-2-oxo-1,3-oxazinan-3-yl}propyl)thiophene-2-carboxylate;
5-(3-{(4S)-4-[(3R)-4-(3,5-dichlorophenyl)-4,4-difluoro-3-hydroxybutyl]-2-oxo-1,3-oxazinan-3-yl}propyl)thiophene-2-carboxylic acid;
Isopropyl 5-(3-{(4R)-4-[(1E,3R)-4-(3,5-dimethylphenyl)-4,4-difluoro-3-hydroxybut-1-en-1-yl]-2-oxo-1,3-oxazinan-3-yl}propyl)thiophene-2-carboxylate;
5-(3-{(4R)-4-[(1E,3R)-4-(3,5-dimethylphenyl)-4,4-difluoro-3-hydroxybut-1-en-1-yl]-2-oxo-1,3-oxazinan-3-yl}propyl)thiophene-2-carboxylic acid;
Isopropyl 5-(3-{(4S)-4-[(3R)-4-(3,5-dimethylphenyl)-4,4-difluoro-3-hydroxybutyl]-2-oxo-1,3-oxazinan-3-yl}propyl)thiophene-2-carboxylate;
5-(3-{(4S)-4-[(3R)-4-(3,5-dimethylphenyl)-4,4-difluoro-3-hydroxybutyl]-2-oxo-1,3-oxazinan-3-yl}propyl)thiophene-2-carboxylic acid;
Isopropyl 5-[3-((4R)-4-{(1E,3R)-4,4-difluoro-3-hydroxy-4-[3-(trifluoromethyl)phenyl]but-1-en-1-yl}-2-oxo-1,3-oxazinan-3-yl)propyl]thiophene-2-carboxylate;
5-[3-((4R)-4-{(1E,3R)-4,4-difluoro-3-hydroxy-4-[3-(trifluoromethyl)phenyl]but-1-en-1-yl}-2-oxo-1,3-oxazinan-3-yl)propyl]thiophene-2-carboxylic acid;
Isopropyl 5-[3-((4S)-4-{(3R)-4,4-difluoro-3-hydroxy-4-[3-(trifluoromethyl)phenyl]butyl}-2-oxo-1,3-oxazinan-3-yl)propyl]thiophene-2-carboxylate;
5-[3-((4S)-4-{(3R)-4,4-difluoro-3-hydroxy-4-[3-(trifluoromethyl)phenyl]butyl}-2-oxo-1,3-oxazinan-3-yl)propyl]thiophene-2-carboxylic acid;
5-(3-{(4R)-4-[(1E,3R)-4,4-difluoro-3-hydroxy-4-(3-isopropylphenyl)but-1-en-1-yl]-2-oxo-1,3-oxazinan-3-yl}propyl)thiophene-2-carboxylic acid;
5-(3-{(4S)-4-[(3R)-4,4-difluoro-3-hydroxy-4-(3-isopropylphenyl)butyl]-2-oxo-1,3-oxazinan-3-yl}propyl)thiophene-2-carboxylic acid;
Methyl 4-bromo-5-(3-{(4R)-4-[(1E,3R)-4-(3-bromophenyl)-4,4-difluoro-3-hydroxybut-1-en-1-yl]-2-oxo-1,3-oxazinan-3-yl}propyl)thiophene-2-carboxylate;
4-Bromo-5-(3-{(4R)-4-[(1E,3R)-4-(3-bromophenyl)-4,4-difluoro-3-hydroxybut-1-en-1-yl]-2-oxo-1,3-oxazinan-3-yl}propyl)thiopbene-2-carboxylic acid;
5-(3-{(4R)-4-[(1E,3R)-4-(3-bromophenyl)-4,4-difluoro-3-hydroxybut-1-en-1-yl]-2-oxo-1,3-oxazinan-3-yl}propyl)-3-fluorothiophene-2-carboxylic acid;
5-(3-{(4S)-4-[(3R)-4-(3-bromophenyl)-4,4-difluoro-3-hydroxybutyl]-2-oxo-1,3-oxazinan-3-yl}propyl)-3-fluorothiophene-2-carboxylic acid; and
5-(3-{(4S)-4-[(3R)-3-(4′-chloro-2′-methylbiphenyl-3-yl)-3-hydroxypropyl]-2-oxo-1,3-oxazinan-3-yl}propyl)thiophene-2-carboxylic acid;
Isopropyl 5-(3-{(4R)-4-[(1E,3R)-4-(3-bromo-5-iodophenyl)-4,4-difluoro-3-hydroxybut-1-en-1-yl]-2-oxo-1,3-oxazinan-3-yl}propyl)thiophene-2-carboxylate; and
5-(3-{(4R)-4-[(1E,3R)-4-(3-bromo-5-iodophenyl)-4,4-difluoro-3-hydroxybut-1-en-1-yl]-2-oxo-1,3-oxazinan-3-yl}propyl)thiophene-2-carboxylic acid;
or a pharmaceutically acceptable salt, enantiomer, diastereomer, prodrug or mixture thereof.

9. A compound according to claim 7 which is:
isopropyl 5-(3-{(4R)-4-[(1E)-4-(3-bromophenyl)-4,4-difluoro-3-oxobut-1-en-1-yl]-2-oxo-1,3-oxazinan-3-yl}propyl)thiophene-2-carboxylate;
isopropyl 5-(3-{(4R)-4-[(1E)-4-(3-bromophenyl)-4,4-difluoro-3-hydroxybut-1-en-1-yl]-2-oxo-1,3-oxazinan-3-yl}propyl)thiophene-2-carboxylate;
isopropyl 5-(3-{(4R)-4-[(1E,3R)-4-(3-bromophenyl)-4,4-difluoro-3-hydroxybut-1-en-1-yl]-2-oxo-1,3-oxazinan-3-yl}propyl)thiophene-2-carboxylate;
5-(3-{(4R)-4-[(1E,3R)-4-(3-bromophenyl)-4,4-difluoro-3-hydroxybut-1-en-1-yl]-2-oxo-1,3-oxazinan-3-yl}propyl)thiophene-2-carboxylic acid;
5-(3-{(4S)-4-[(3R)-4-(3-bromophenyl)-4,4-difluoro-3-hydroxybutyl]-2-oxo-1,3-oxazinan-3-yl}propyl)thiophene-2-carboxylic acid;
5-(3-{(4R)-4-[(1E)-4,4-difluoro-3-hydroxy-4-phenylbut-1-en-1-yl]-2-oxo-1,3-oxazinan-3-yl}propyl)thiophene-2-carboxylic acid;
or a pharmaceutically acceptable salt, enantiomer, diastereomer, prodrug or mixture thereof.

10. A pharmaceutical composition comprising a pharmaceutically acceptable carrier and a compound of formula I, as recited in claim 1.

11. Use of a compound of claim 1 for the manufacture of a medicament for treating ocular hypertension or glaucoma.

12. The composition according to claim 10 wherein one or more active ingredients belonging to the group consisting of: β-adrenergic blocking agent, parasympatho-mimetic agent, sympathomimetic agent, carbonic anhydrase inhibitor, Maxi-K channel blocker, and a prostaglandin, hypotensive lipid, neuroprotectant, and 5-HT2 receptor agonist is optionally added.

13. The composition according to claim 12 wherein the β-adrenergic blocking agent is timolol, betaxolol, levobetaxolol, carteolol, or levobunolol; the parasympathomimetic agent is pilocarpine; the sympathomimetic agent is epinephrine, brimonidine, iopidine, clonidine, or para-aminoclonidine, the carbonic anhydrase inhibitor is dorzolamide, acetazolamide, metazolamide or brinzolamide; COSOPT®, the Maxi-K is Penitrem A, paspalicine, charybdotoxin, iberiotoxin, Paxicillan, Aflitram, Verroculogen, 1-(1-isobutyl-6-methoxy-1H-indazol-3-yl)-2-methylpropan-1-one; 1-[1-(2,2-dimethylpropyl)-6-methoxy-1H-indazol-3-yl]-2-methylpropan-1-one; 1-[1-(cyclohexylmethyl)-6-methoxy-1H-indazol-3-yl]-2-methylpropan-1-one; 1-(1-hexyl-6-methoxy-1H-indazol-3-yl)-2-methylpropan-1-one; 1-[1-(2-ethylhexyl)-6-methoxy-1H-indazol-3-yl]-2-methylpropan-1-one; 1-(3-isobutyryl-6-methoxy-1H-indazol-1-yl)buan-2-one; 1-(3-isobutyryl-6-methoxy-1H-indazol-1-yl)-3,3-dimethylbutan-2-one; 1-(3-cyclopentylcarbonyl)-6-methoxy-1H-indazol-1-yl)-3,3-dimethylbutan-2-one; 1-(3,3-dimethyl-2-oxobutyl)-6-methoxy-1H-indazole-3-carboxylic acid; and 1-[3-(3-hydroxypropanoyl)-6-methoxy-1H-indazol-1-yl]-3,3-dimethylbutan-2-one, the prostaglandin is latanoprost, travaprost, unoprostone, rescula, or S1033, the hypotensive lipid is lumigan, the neuroprotectant is eliprodil, R-eliprodil or memantine; and the 5-HT2 receptor agonist is 1-(2-aminopropyl)-3-methyl-1H-imdazol-6-ol fumarate or 2-(3-chloro-6-methoxy-indazol-1-yl)-1-methyl-ethylamine.

14. The composition according to claim 10 which is a topical formulation in the form of a solution or suspension, said composition optionally containing xanthan gum or gellan gum.

15. Use of a compound of claim 1 for the manufacture of a medicament for treating macular edema or macular degeneration, treating dry eye, increasing retinal and optic nerve head blood velocity, increasing retinal and optic nerve oxygen tension or providing a neuroprotection.

16. Use of a compound of claim 1 for the manufacture of a medicament for stimulating bone formation, treating or reducing the risk of contracting a disease state or condition related to abnormal bone resorption, in a mammal in need thereof.
